(19) United States
(12) Patent Application Publication
(10) Pub. No.: US 2025/0100027 A1
(43) Pub. Date: Mar. 27, 2025

(54) DEVICE AND SYSTEM FOR CLEANING MEDICAL DEVICES WITH INFLATABLE COMPONENTS AND METHODS THEREOF

(71) Applicant: INNOVATIVE HEALTH, SCOTTSDALE, AZ (US)

(72) Inventors: Haydn Oleson, Peoria, AZ (US); Blessan C. Joseph, Chandler, AZ (US); Rafal Chudzik, Peoria, AZ (US); Emigdio Esquivel, Scottsdale, AZ (US)

(21) Appl. No.: 18/896,140

(22) Filed: Sep. 25, 2024

Related U.S. Application Data
(60) Provisional application No. 63/540,204, filed on Sep. 25, 2023.

(51) Int. Cl. B08B 9/00; A61B 90/70; A61M 25/00; A61M 25/10

(52) U.S. Cl. CPC B08B 9/00; A61B 90/70; A61B 2090/701; A61M 2025/0019; A61M 25/104

(57) ABSTRACT

Methods, devices, and systems for cleaning medical devices with inflatable components are provided herein. The methods, devices, and systems can be used to clean the exterior, the lumen, and the inflatable component of a medical device. The system can include a cleaning solution source, at least one medical device attachment port, and at least one pressure source. The cleaning solution source can be operable to contain a fluid. The at least one medical device attachment port can be in fluid communication with the inflatable component. The at least one pressure source can be operable to move the fluid from the cleaning solution source to the at least one medical device attachment port.

### CROSS-REFERENCE TO RELATED APPLICATIONS

This application claims the benefit of U.S. Application No. 63/540,204, filed Sep. 25, 2023, the entire contents of which are incorporated herein by reference in their entirety.

### FIELD OF THE INVENTION

The present disclosure provides methods, devices, and systems for cleaning medical devices with inflatable components.

### BACKGROUND OF THE INVENTION

Single-use Percutaneous Transluminal Angioplasty (PTA) devices are catheters that have an inflatable component attached to the distal end of the catheter. The inflatable component can be pressurized using a contrast/saline mixture. These devices are used throughout the vasculature of the human body, therefore, increasing the associated risk to the patient when using these devices. Currently, there are no known medical device reprocessing companies that have undertaken the task to reprocess PTA devices due to their high-risk profiles. The inflatable components are made of various polymers and materials that allow them to withstand high pressures, and some lower pressures. Nonetheless, these inflatable components can be fragile if not handled correctly. This makes the reprocessing strategy more difficult.

### SUMMARY OF THE INVENTION

Provided herein is a system for cleaning a medical device that include an inflatable component. The system can include a cleaning solution source operable to contain a fluid, at least one medical device attachment port in fluid communication with the inflatable component, and at least one pressure source operable to move the fluid from the cleaning solution source to the at least one medical device attachment port. In at least one example, the at least one medical device attachment port can include a cleaning valve. In at least one example, the cleaning valve can be operable to direct the fluid into the inflatable component and direct the fluid out of the inflatable component.

In at least one example, the system can include at least one pressure transducer and/or pressure sensor operable to measure a pressure inside the inflatable component. In at least one example, the system can include one or more fluidic paths that include an inflatable component fluidic path, a lumen fluidic path, and an exterior fluidic path. In at least one example, the inflatable component fluidic path can be operable to provide the fluid to the inflatable component, the lumen fluidic path can be operable to provide the fluid to one or more lumens of the medical device, and the exterior fluidic path can be operable to provide the fluid to an exterior surface of the medical device. In at least one example, the at least one medical device attachment port can include a plurality of medical device attachment ports.

In at least one example, the system can include one or more manifolds in fluid communication with the at least one pressure source. In at least one example, the one or more manifolds can be operable to direct the fluid to the plurality of medical device attachment ports. In at least one example, the system can include at least one fill sensor operable to determine that an entirety of an interior surface of the inflatable component has been contacted with the fluid. In at least one example, the at least one fill sensor can include a pressure sensor, a pressure transducer, a force gauge system, a proximity sensor system, a scale, a flow rate sensor, a machine vision system, and/or a laser micrometer system.

Further provided herein is a system for cleaning a medical device with an inflatable component. The system can include one or more fluidic paths including an inflatable component fluidic path, a lumen fluidic path, and an exterior fluidic path. The system can include at least one cleaning solution source in fluid communication with the one or more fluidic paths. The cleaning solution source can be operable to contain a fluid. The system can include at least one pressure source operable to direct the fluid to the one or more fluidic paths. The system can include at least one medical device attachment port in fluid communication with the inflatable component fluidic path. The system can include one or more valves in fluid communication with the one or more fluidic paths. The one or more valves can be operable to direct fluid to the inflatable component fluidic path, the lumen fluidic path, and the exterior fluidic path.

In at least one example, the system can include at least one inflatable component fill sensor. The inflatable component fill sensor can be operable to determine an interior surface of the inflatable component is fully contacted with the fluid. In at least one example, the system can include at least one lumen pressure sensor and/or pressure transducer. The at least one lumen pressure sensor and/or pressure transducer can be operable to measure a pressure within a lumen of the medical device. In at least one example, the at least one medical device attachment port can include a plurality of medical device attachment ports. In at least one example, the system can include one or more manifolds in fluid communication with the one or more fluidic paths. The one or more manifolds can be operable to direct the fluid to the plurality of medical device attachment ports.

Further provided herein is a method for cleaning a medical device with an inflatable component. The method can include coupling the medical device with the inflatable component to the at least one medical device attachment port, providing, via at least one pressure source, a fluid to the inflatable component through the at least one medical device attachment port, and monitoring, via at least one fill sensor, a level of contact of the fluid with an interior surface of the inflatable component.

In at least one example, the method can include turning off the at least one pressure source when the level of contact indicates an entirety of the interior surface of the inflatable component is contacted with the fluid. In at least one example, the method can further include determining whether the inflatable component has a leak based on a rate of change in the level of contact. In at least one example, the method can include removing, via the at least one pressure source, the fluid from the inflatable component, and monitoring, via a chemical sensor, an iodine concentration in the fluid removed from the inflatable component. In at least one example, the method can include repeating the method when the iodine concentration indicates the inflatable component has not been sufficiently cleaned of contrast.

Further provided herein is a method for cleaning one or more medical devices that include an inflatable component. The method can include coupling the one or more medical devices to at least one medical device attachment port; actuating one or more valves in communication with at least one pressure source such that the inflatable component is in communication with the at least one pressure source and a cleaning solution source; providing a fluid to the inflatable component via the cleaning solution source and the at least one pressure source; actuating the one or more valves such that the fluid is retained in the inflatable component for a dwell period; actuating the one or more valves such the inflatable component is in communication with a contamination sink and the at least one pressure source; and removing the fluid from the inflatable component to the contamination sink via the at least one pressure source.

In at least one example, the method can include monitoring a pressure inside the inflatable component when the fluid is retained in the inflatable component. In at least one example, when the pressure decreases by a threshold amount during the dwell period, the inflatable component can be determined to have a leak. In at least one example, the one or more medical devices can include a plurality of medical devices. In at least one example, the one or more valves include a plurality of valves operable to selectively supply the fluid to each of the plurality of medical devices. In at least one example, the fluid includes one or more of micro-pellets, microbeads, microparticles, abrasive particles, and/or microbubbles operable to provide abrasive cleaning to the inflatable component and/or a lumen of the one or more medical devices.

### BRIEF DESCRIPTION OF THE FIGURES

Details of one or more aspects of the subject matter described in this disclosure are set forth in the accompanying drawings and the description below. However, the accompanying drawings illustrate only some typical aspects of this disclosure and are therefore not to be considered limiting of its scope. Other features, aspects, and advantages will become apparent from the description, the drawings and the claims.

FIG. 1 illustrates a system for cleaning medical devices with inflatable components.

FIG. 2 illustrates a system for cleaning medical devices with inflatable components.

FIG. 3 illustrates a system for cleaning medical devices with inflatable components.

FIG. 4 depicts a force gauge sensor system.

FIG. 5 depicts a proximity sensor system.

FIG. 6A depicts a scale system.

FIG. 6B depicts a scale system.

FIG. 7 depicts a scale system.

FIG. 8A depicts internal and external pressures on a medical device with a inflatable component.

FIG. 8B depicts internal and external pressures on a medical device with a inflatable component.

FIG. 9 depicts a system for cleaning medical devices with inflatable components.

FIG. 10 depicts a system for cleaning medical devices with inflatable components.

FIGS. 11A-11C depict a flow chart for a method of cleaning medical devices with inflatable components.

FIG. 12 depicts a cleaning process flow chart.

FIG. 13 is a schematic diagram of a controller.

FIG. 14 depicts a method for cleaning a medical device with a inflatable component.

FIG. 15 depicts a machine vision system.

FIGS. 16A-16B depict a laser micrometer system.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the examples described herein. However, it will be understood by those of ordinary skill in the art that the examples described herein can be practiced without these specific details. In other instances, methods, procedures, and components have not been described in detail so as not to obscure the related relevant feature being described. Also, the description is not to be considered as limiting the scope of the embodiments described herein. The drawings are not necessarily to scale and the proportions of certain parts may be exaggerated to better illustrate details and features of the present disclosure.

Several definitions that apply throughout the above disclosure will now be presented.

The term “coupled” is defined as connected, whether directly or indirectly through intervening components, and is not necessarily limited to physical connections. The connection can be such that the objects are permanently connected or releasably connected.

The term “substantially” is defined to be essentially conforming to the particular dimension, shape or other word that substantially modifies, such that the component need not be exact.

The terms “comprising,” “including” and “having” are used interchangeably in this disclosure. The terms “comprising,” “including” and “having” mean to include, but not necessarily be limited to the things so described.

As used herein, “about” refers to numeric values, including whole numbers, fractions, percentages, etc., whether or not explicitly indicated. The term “about” generally refers to a range of numerical values, for instance, +0.5-1%, +1-5% or +5-10% of the recited value, that one would consider equivalent to the recited value, for example, having the same function or result.

The present disclosure relates to devices, systems, and methods for cleaning medical devices with inflatable components. In some examples, the inflatable components can be balloons attached to catheters. The devices, systems, and methods can be operable to clean contrast, dirt, and/or other materials from medical devices after use and/or after manufacture. In some examples, the devices, systems, and methods allow for sufficient reprocessing of medical devices with inflatable components such that medical devices with inflatable components can be reused in a patient. For example, after reprocessing, the medical devices with the inflatable component can be sufficiently cleaned to pass safety and health regulations. In some examples, the devices, systems, and methods can clean the inflatable component, exterior, and one or more lumens of the medical device. The devices, systems, and methods can also be operable to test the medical devices for deficiencies such as leaks or other defects, such that defective devices are not reused in a patient.

The system can include one or more fluidic paths operable to clean different components of a medical device that includes an inflatable component. In some examples, the one or more fluidic paths can include a plurality of fluidic paths. In some examples, the one or more fluidic paths can be configured to clean an inflatable component (e.g., via inflation lumen) of a medical device. In some examples, the one or more fluidic paths can be configured to clean a lumen (e.g., a guidewire lumen) of a medical device. In some examples, the one or more fluidic paths can be configured to clean an exterior of the medical device. In some examples, a first fluidic path can be operable to clean the inflatable component (e.g., via inflation lumen) of the medical device. In some examples, a second fluidic path can be operable to clean a lumen (e.g., guidewire lumen) of a medical device. In some examples, a third fluidic path can be operable to clean an exterior of the medical device. In some examples, one fluidic path can be operable to clean all of the components of the medical device. In some examples, the medical device can be checked (e.g., validated) after cleaning is completed to ensure the medical device is safe for use in a patient.

The system can include one or more fluidic paths, a cleaning solution source, at least one medical device attachment port, and at least one pressure source. In some examples, the cleaning solution source can be in fluid communication with the one or more fluidic paths. The cleaning solution source can be operable to contain a fluid. The fluid can be delivered to the inflatable component via the one or more fluidic paths. In some examples, the at least one medical device attachment port can be in fluid communication with the one or more fluidic paths and the inflatable component of the medical device. In some examples, the at least one pressure source can be in fluid communication with the one or more fluidic paths. The at least one pressure source can be operable to move the fluid through the one or more fluidic paths. The system can be operable to provide a fluid (e.g., cleaning solution) to the inflatable component, empty the fluid (e.g., cleaning solution from the inflatable component), test the inflatable component for leaks after the completion of a cleaning process, and determine whether the inflatable component has any remaining contrast (e.g., dirt, debris, and/or other material from previous use) above a predetermined threshold. The system can be automated (e.g., function without user input) and configured to detect when the inflatable component is free from contrast. If the medical device has any remaining contrast above a predetermined threshold, the system can start the cleaning process again (e.g., move the fluid through the fluidic paths, provide the fluid to the inflatable component, etc.).

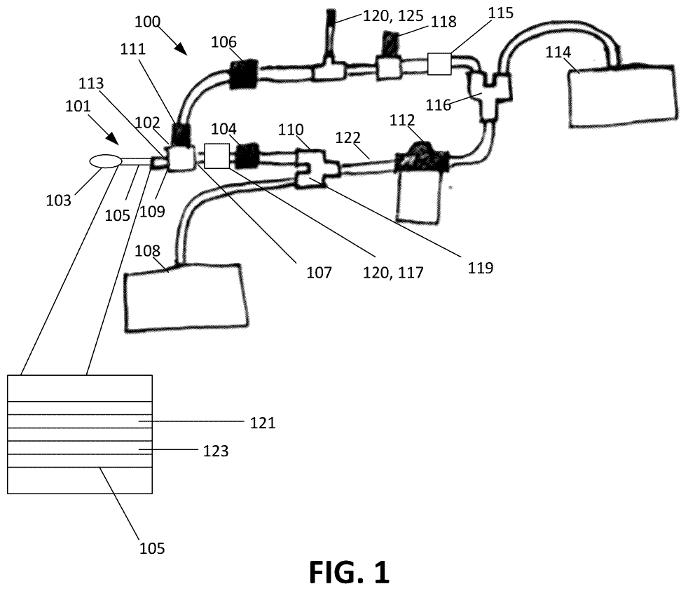

FIG. 1 illustrates the system 100 for cleaning a medical device 101 with an inflatable component 103. The medical device 101 can include a catheter body 105. The catheter body can include one or more lumens. In some examples, the catheter body 105 can include an inflation lumen 121 in communication with the inflatable component 103 and one or more additional lumens 123. In some examples, the system 100 for cleaning the medical device 101 with an inflatable component 103 can be operable to clean a plurality of medical devices 101 with inflatable components 103.

The system 100 can include one or more fluidic paths 122 operable to fluidically connect at least one medical device attachment port 102 to a cleaning solution source 108. In some examples, the one or more fluidic paths 122 can also fluidically connect the at least one medical device attachment port 102 to a contamination sink 114. The at least one medical device attachment port 102 can be operable to fluidically connect a lumen (e.g., inflation lumen 121), and thereby an inflatable component 103, of the medical device 101 to the one or more fluidic paths 122. The at least one medical device attachment port 102 can include an inlet port 107 (e.g., cleaning valve inlet port 107 operable to receive a fluid (e.g., cleaning solution) from the cleaning solution source 108 through the one or more fluidic paths 122. The at least one medical device attachment port 102 can include a first outlet port 109 operable to deliver the fluid (e.g., cleaning solution) to a lumen (e.g., inflation lumen 121) in fluid communication with an inflatable component 103 of a medical device 101. In some examples, the at least one medical device attachment port 102 can include a second outlet operable to expel the fluid (e.g., cleaning solution) from the lumen of the inflatable component 103 to a contamination sink 114 fluidically attached to the one or more fluidic paths 122. In some examples, the first outlet port 109 of the at least one medical device attachment port 102 can be operable to both deliver the fluid to the lumen and receive fluid from the lumen.

The one or more fluidic paths 122 can include conduits, tubing, or other materials operable to form fluid flow pathways. The one or more fluidic paths 122 can include one conduit, tubing, etc. to form the fluidic path 122. In some examples, the fluidic paths 122 can include a plurality of conduits, tubing, etc. coupled together to form the fluid flow pathways.

The system 100 can further include at least one pressure source 112 (e.g., first pump 112). The at least one pressure source 112 can be in fluid communication with the cleaning solution source 108, the at least one medical device attachment port 102, and the contamination sink 114. The at least one pressure source 112 can be operable to move (e.g., direct and/or circulate) the fluid (e.g., cleaning solution) from the cleaning solution source 108 to the inlet of the at least one medical device attachment port 102 and into the lumen (e.g., inflation lumen 121 of the medical device 101, and thereby deliver the fluid to the inflatable component 103. The at least one pressure source can be operable to move (e.g., direct and/or circulate) the fluid from the at least one medical device attachment port 102 (e.g., via the lumen and the inflatable component 103) to the contamination sink 114. The system 100 can further include one or more fill sensors 120 in fluid communication with the one or more fluidic paths 122. The one or more fill sensors 120 can be operable to determine a level of contact of the fluid with an interior surface of the inflatable component 103.

In some examples, the cleaning solution source 108 can include a microbubble generator, microbubble emitter, microbubble diffuser, and/or microbubble system operable to create microbubbles in the fluid (e.g., cleaning solution). The microbubbles can provide abrasive cleaning (e.g., when the bubbles pop) to the interior of the inflatable component 103 and/or lumen 121, 123 as the fluid is provided to the inflatable component 103 and/or lumen 121, 123 as described herein.

As illustrated in FIGS. 1-3, the system 100 can include one or more fluidic paths 122 and a variety of components to clean the inflatable component 103. The system 100 can include at least one medical device attachment port 102, a first manifold 104 (e.g., medical device attachment port inlet manifold), a second manifold 106 (e.g., medical device attachment port outlet manifold), at least one pressure source 112 (e.g., first pump 112), a fluid supply valve 110, a cleaning solution source 108, a contamination valve 116, a contamination sink 114, an open/close valve 118, and a fill sensor 120 (e.g., pressure transducer 125). All of the components of the system 100 can be fluidically connected to the one or more fluidic paths 122. In some examples, the system 100 can be fully automated by a controller. In some examples, the open/close valve 118 can include a solenoid valve.

In some examples, the at least one medical device attachment port 102 can include a cleaning valve. In some examples, the at least one medical device attachment port 102 can include a diverter valve. In some examples, the at least one medical device attachment port 102 can include a three-way valve. In some examples, the at least one medical device attachment port 102 can include a two-way valve. The at least one medical device attachment port 102 can have an inlet port 107 operable to receive a fluid (e.g., cleaning solution) from the cleaning solution source 108. In some examples, the at least one medical device attachment port 102 can include a first outlet port 109. In some examples, the first outlet port 109 can be operable to provide the fluid to the lumen (e.g., inflation lumen 121) and thereby the inflatable component 103 of the medical device 101. In some examples, the first outlet port 109 can couple directly to the lumen (e.g., inflation lumen 121) of the medical device 101 with the inflatable component 103. In some examples, the first outlet port 109 can be coupled to a connector 113 (e.g., Luer connector). The connector 113 can be operable to removably couple to the lumen (e.g., inflation lumen 121) of the medical device 101 with the inflatable component 103. In some examples, the at least one medical device attachment port 102 can have a second outlet port 111. The second outlet port 111 can be operable to receive fluid from the inflatable component 103, via the inflation lumen 121, thereby expelling the fluid from the inflatable component 103.

The at least one pressure source 112 (e.g., first pump 112) can provide a pressure to move the fluid (e.g., cleaning solution) from the cleaning solution source 108 through the first manifold 104 and into the at least one medical device attachment port 102. The at least one pressure source 112 can include any pressure source operable to provide a pressure to move the fluid from the cleaning solution source 108 to the at least one medical device attachment port 102.

The cleaning solution source 108 can be fluidically connected to the one or more fluidic paths 122 via a fluid supply valve 110. The fluid supply valve 110 can be operable to provide the fluid (e.g., cleaning solution) to the one or more fluidic paths 122 when the system 100 is in a cleaning mode of operation. In some examples, the fluid can include a cleaning solution. In some examples, the cleaning solution can include any cleaning solution capable of cleaning an inflatable component 103 of a medical device 101 (e.g., by breaking down contrast in the inflatable component 103 from previous use or manufacturing). The at least one pressure source 112 (e.g., first pump 112) can be turned on and pull the fluid from the cleaning solution source 108 and into the one or more fluidic paths 122. The fluid can then travel to the at least one medical device attachment port 102 through the one or more fluidic paths 122 via the pressure provided by the at least one pressure source 112 (e.g., first pump 112). When the system 100 is in a contaminated fluid removal mode, the fluid supply valve 110 can close the inlet 119 to the cleaning solution source 108 such that no fluid is supplied to the one or more fluidic paths 122. Additionally, no contaminated fluid can flow into the cleaning solution source 108.

The one or more fluidic paths 122 illustrated in FIG. 1 can flow in a certain direction. The at least one pressure source 112 (e.g., first pump 112) is turned on to provide a pressure to the one or more fluidic paths 122. Fluid (e.g., cleaning solution) is pulled from the cleaning solution source 108 via negative pressure created by the at least one pressure source 112 (e.g., first pump 112) and through the fluid supply valve 110. The fluid can then flow through the first manifold 104 to the at least one medical device attachment port 102. The fluid can be provided to the inflatable component 103 via the lumen (e.g., inflation lumen 121) coupled to the at least one medical device attachment port 102. In some examples, the at least one pressure source 112 (e.g., first pump 112) can be operable to provide a negative pressure (e.g., vacuum pressure) to the one or more fluidic paths 122. The fluid can be pulled from the inflatable component 103, via the negative pressure, at the at least one medical device attachment port 102 through the second manifold 106, the fill sensor 120 (e.g., pressure transducer 125), and the open/close valve 118. The contamination valve 116 can be operable to provide fluid to the contamination sink 114 when the at least one pressure source 112 is providing a negative pressure. The contamination valve 116 can be operable to allow continuous circulation of fluid when the at least one pressure source 112 is providing a positive pressure.

The inlet port 107, first outlet port 109, and second outlet port 111 of the at least one medical device attachment port 102 can each have an open state and a closed state. The inlet port 107 can be placed in the open state to allow fluid (e.g., cleaning solution) to enter the inflatable component 103 from the cleaning solution source 108 using a supplied pressure from the first pump 112. The first outlet port 109 and second outlet port 111 of the at least one medical device attachment port 102 (e.g., cleaning valve) can be placed in an open state to allow the inflatable component 103 to fill with cleaning solution. The open/close valve 118 can be placed in a closed state such that the fluid (e.g., cleaning solution) fills the inflatable component 103 and a pressure inside the inflatable component 103 can be measured via the pressure transducer 125. When the open/close valve 118 is placed in the closed state, pressure builds up in the inflatable component 103. Once the pressure inside the inflatable component 103 reaches a desired pressure as measured by the pressure transducer 125, the first pump 112 can be turned off such that no additional pressure is provided. In an aspect, the pressure transducer 125 measures the pressure along the one or more fluidic paths 122 between the first pump 112 and the open/close valve 118, which includes the pressure inside the inflatable component 103. In some examples, the controller can be configured to turn the first pump 112 off when the inflatable component 103 reaches the desired pressure. As described further herein, other types of fill sensors 120 can be used in conjunction with, or as alternatives to, the pressure transducer 125. The controller can be configured to turn the first pump 112 off when any of the fill sensors 120 described herein indicate that the inflatable component 103 is fully inflated (e.g., the fluid has contacted the entire interior surface of the inflatable component 103).

In some examples, the pressure supplied by the at least one pressure source 112 (e.g., first pump 112) can be a nominal pressure. The supplied pressure can be about 10 psi to about 20 psi, about 20 psi to about 30 psi, about 30 psi to about 40 psi, about 40 psi to about 50 psi, about 50 psi to about 60 psi, about 60 psi to about 70 psi, about 70 psi to about 80 psi, about 80 psi to about 90 psi, about 90 psi to about 100 psi, or more.

In some examples, the desired pressure can be a pressure that ensures expansion of the inflatable component 103 greater than a predetermined expansion threshold. For example, the inflatable component 103 can be expanded such that the entirety of the fibers and/or material is exposed. In some examples, the inflatable component 103 can be fully expanded without breaking so that the inflatable component 103 can return to the pre-expanded configuration and retain elasticity. When the inflatable component 103 is expanded, contact of the cleaning solution with the full interior surface area of the inflatable component 103 is ensured. The desired pressure of the inflatable component 103 can be calculated depending on the type of inflatable component 103 (e.g., compliant, semi-compliant, or non-compliant). Eq. 1 can be used to determine an outer diameter of the inflated inflatable component 103, and therefore full contact of the interior surface area of the inflatable component 103.

$$Compliance Assessment = \frac{OD @ RBP - OD @ NP}{OD @ RBP} \times 100 \% \qquad ( Eq . 1 )$$

For an inflatable component 103 having a known compliance, a known rated burst pressure (RBP), and a known outer diameter (OD) at the rated burst pressure, the necessary outer diameter at a nominal pressure can be calculated. The outer diameter at the nominal pressure can then be used to determine the nominal pressure necessary to inflate the inflatable component 103 with a sufficient amount of fluid (e.g., cleaning solution) to ensure full contact of the fluid (e.g., cleaning solution) with the entire interior surface area of the inflatable component 103. In some examples, the desired pressure can be below an operating pressure of the inflatable component 103. In examples, the desired pressure can be the minimum pressure to allow the inflatable component 103 to experience a minimal change in diameter. The desired pressure can be a pressure that allows the inflatable component 103 to experience a change in diameter. When the inflatable component 103 experiences a change in diameter above the predetermined expansion threshold, the entire interior surface of the inflatable component 103 is in contact with the cleaning solution.

In some examples, once the inflatable component 103 has reached the desired pressure, and therefore the cleaning solution is in contact with the entire interior surface area of the inflatable component 103, the at least one pressure source 112 (e.g., first pump 112) can be turned off. The fluid (e.g., cleaning solution) can be allowed to dwell in the inflatable component 103 for a dwell period. In an aspect, the dwell period can be a sufficient amount of time for the fluid (e.g., cleaning solution) to break down contrast inside the inflatable component 103. In some examples, the dwell period can be about 1 second to about 5 seconds, about 5 seconds to about 10 seconds, about 10 seconds to about 15 seconds, about 15 seconds to about 20 seconds, about 20 seconds to about 25 seconds, about 25 seconds to about 30 seconds, about 30 seconds to about 35 seconds, about 35 seconds to about 40 seconds, about 40 seconds to about 45 seconds, about 45 seconds to about 50 seconds, about 50 seconds to about 55 seconds, about 55 seconds to about 60 seconds, about 1 minute to about 1.5 minutes, about 1.5 minutes to about 2 minutes, about 2 minutes to about 2.5 minutes, about 2.5 minutes to about 3 minutes, about 3 minutes to about 3.5 minutes, about 3.5 minutes to about 4 minutes, about 4 minutes to about 4.5 minutes, about 4.5 minutes to about 5 minutes, or more.

In some examples, the fluid (e.g., cleaning solution) can be operable to break down contrast inside the inflatable component 103. Contrast, such as saline and other chemicals, can build up over time after use in a human patient. The contrast can crystallize and become hard. The fluid (e.g., cleaning solution) can be configured such that it breaks down the contrast in the inflatable component 103. In some examples, the fluid can include micro-pellets, microbeads, microparticles, and/or abrasive particles suspended in the fluid. The micro-pellets, microbeads, microparticles, and/or abrasive particles can provide abrasive cleaning to the inflatable component 103 and/or lumen 121, 123 when the fluid including the micro-pellets, microbeads, microparticles, and/or abrasive particles are provided to the inflatable component 103 and/or lumen 121, 123 as described herein.

In some examples, the system 100 can be operable to cause the fluid inside the inflatable component 103 to move to ensure that fresh fluid continues contacting the interior surface of the inflatable component 103. In some examples, the movement of the fluid can provide a physical force to remove the contrast and/or material inside the inflatable component 103 from the interior surface. In some examples, the system can include a rocker plate or other movement mechanism to physically move the inflatable component 103 when it is filled with fluid.

After the dwell period, the system 100 can empty the inflatable component 103 of contaminated fluid through the one or more fluidic paths 122. The inlet port 107 of the at least one medical device attachment port 102 can be placed in the closed state and the open/close valve 118 can be placed in the open state. The first outlet port 109 and the second outlet port 111 of the at least one medical device attachment port 102 can be placed in the open state to remove the contaminated fluid. The at least one pressure source 112 (e.g., first pump 112) can be switched to a different mode of operation to provide a vacuum pressure to the one or more fluidic paths 122. The contaminated fluid can be pulled out of the inflatable component 103 by the vacuum pressure and flow to the contamination sink 114 through the one or more fluidic paths 122. In some examples, the controller can automatically switch the at least one pressure source 112 (e.g., first pump 112) to a vacuum operation mode after the dwell time has completed. The controller can also open the first and second outlets of the cleaning valve and close the inlet port 107 of the cleaning valve.

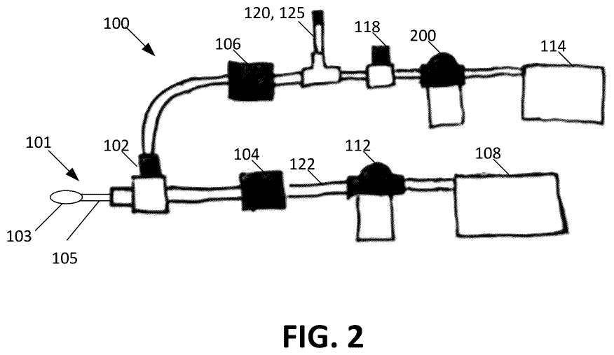

FIG. 2 illustrates the system 100 in some examples. The at least one pressure source 112 can include two pressure sources (e.g., first pump 112 and second pump 200). The first pump 112 can be turned on and pull fluid (e.g., cleaning solution) from the cleaning solution source 108. The fluid can flow through the one or more fluid paths 122 through the first manifold and into the at least one medical device attachment port 102 where the fluid is provided to the inflatable component 103 via the lumen (e.g., inflation lumen 121) of the medical device 101. Once the inflatable component 103 has been cleaned, the first pump 112 can be turned off and a second pump 200 can be turned on. The second pump 200 can provide a vacuum pressure to pull the fluid from the at least one medical device attachment port 102 (e.g., cleaning valve) through the second manifold 106, the pressure transducer 125, the open/close valve 118 and into the contamination sink 114.

As illustrated in FIG. 2, the second pump 200 can be operable to create a vacuum pressure to pull the contaminated fluid from the inflatable component 103 through the fluidic path to the contamination sink 114. In this aspect, the first pump 112 can be operable to provide the fluid (e.g., cleaning solution) to the at least one medical device attachment port 102 to inflate the inflatable component 103. When the inflatable component 103 reaches the desired pressure, and therefore the desired amount of fluid, the at least one pressure source 112 (e.g., first pump 112) can be turned off. In some examples, when the dwell period is being observed, the necessary valves can be closed to allow the fluid to remain in the inflatable component 103. After the dwell period, the second pump 200 can be turned on to pull the contaminated fluid from the inflatable component 103 through the one or more fluidic paths 122 to the contamination sink 114.

In some examples, the first pump 112 can be a centrifugal pump, a gear pump, a diaphragm pump, a peristaltic pump, a piston pump, a rotary vane pump, or a lobe pump. The first pump 112 can be powered electrically, pneumatically, hydraulically, or manually. In some examples, the first pump 112 can be operable to supply sufficient pressure to allow the inflatable component 103 to fully expand its interior surface area greater than an expansion threshold to allow for optimal cleaning (e.g., allow cleaning solution to contact the entire interior surface area of the inflatable component 103). High pressures may not be necessary for cleaning purposes because high pressures can add stress to the material of the inflatable component 103. However, as discussed further herein, high pressures can be used to test the inflatable component 103 for a leak.

In some examples, the second pump 200 can include a vacuum pump, venturi pump, liquid or steam powered jet pump, drum pump, diaphragm vacuum pump, rotary vane vacuum pump, liquid ring, and/or hydraulic cylinder. The second pump 200 can be powered electrically, pneumatically, hydraulically, and/or by steam. In some examples, the second pump 200 does not need to achieve high negative pressures within the inflatable component 103. The second pump can be configured to remove the contaminated fluid from the inflatable component 103 to allow fresh fluid (e.g., cleaning solution) to be cycled into the inflatable component 103.

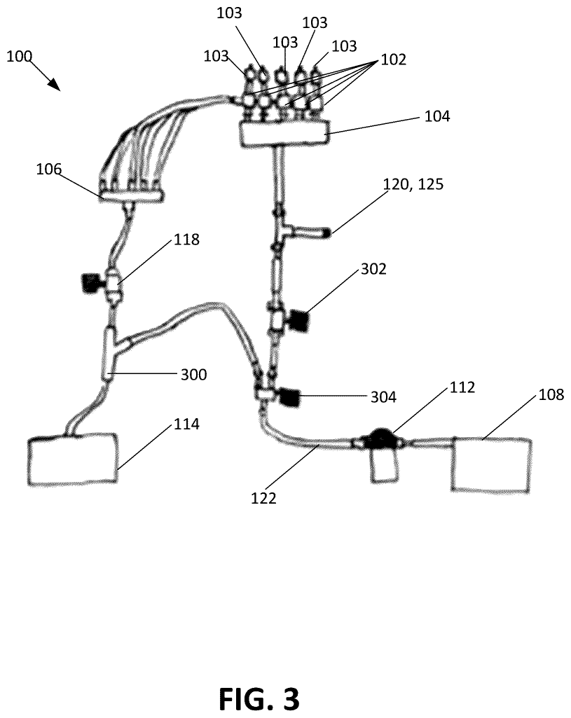

As illustrated in FIG. 3, the system 100 can include a jet pump 300 to pull the contaminated fluid from the inflatable component 103. The jet pump 300 can be in fluid communication with the one or more fluidic paths 122. The jet pump 300 can be upstream of the contamination sink 114. When the system 100 has a jet pump 300, the system 100 can further include a second open/close valve 302 and a pressure supply valve 304. The pressure supply valve 304 can include a diverter valve. The pressure supply valve 304 can be in fluid communication with the one or more fluidic paths 122 downstream from the first pump 112. The first pump 112 can provide pressure to the jet pump 300 to create a vacuum pressure to pull the contaminated fluid from the inflatable component 103. The pressure supply valve 304 can have an inlet that is in an open state whenever pressure is to be supplied to the system 100. The pressure supply valve 304 can have a first outlet port operable to provide a pressure to the at least one medical device attachment port 102 (e.g., cleaning valve). The pressure supply valve 304 can have a second outlet port operable to provide pressure to the jet pump 300. To supply pressure to the inflatable component 103, the second outlet port of the pressure supply valve 304 can be placed in a closed state, the first outlet port of the pressure supply valve 304 can be placed in an open state, and the second open/close valve 302 can be placed in an open state. To supply pressure to the jet pump 300, the second open/close valve 302 can be placed in a closed state and the second outlet port of the pressure supply valve 304 can be placed in an open state, such that a pressure is supplied to the jet pump 300. In some examples, the jet pump 300 can be a venturi pump. In some examples, the pressure supplied to the jet pump can be about 15 psi to about 50 psi, about 50 psi to about 100 psi, about 100 psi to about 150 psi, about 150 psi to about 200 psi, or more.

The first pump 112 can be turned on and the fluid can flow through the one or more fluid paths 122 through a pressure supply valve 304, an open/close valve 302, the pressure transducer 125, and the first manifold 104 into the at least one medical device attachment port 102. To pull fluid from the at least one medical device attachment port 102 (e.g., cleaning valve), pressure supply valve 304 can be actuated such that the pressure from the first pump 112 is diverted to the jet pump 300. The jet pump 300 can provide a negative pressure to pull the contaminated fluid from the at least one medical device attachment port 102, the second manifold 1-6, the open/close valve 118, the jet pump 300, and into the contamination sink 114.

As illustrated in FIG. 1, the contamination sink 114 can be connected to the one or more fluidic paths 122 via a contamination valve 116. The contamination valve 116 can be actuated to allow contaminated fluid to flow out of the one or more fluidic paths 122 and into the contamination sink 114. In some examples, the contamination valve 116 can be actuated such that fluid flows continuously though the one or more fluidic paths 122 and does not flow into the contamination sink 114.

After removing the contaminated fluid, the inflatable component 103 can be provided cleaning solution again in the same manner. The inflatable component 103 can be put through multiple cleaning cycles. In some examples, the inflatable component 103 can undergo 1, 2, 3, 4, 5, 6, 7, 8, 9, 10, 11, 12, 13, 14, 15, 16, 17, 18, 19, 20, or more cycles. In some examples, the type of the inflatable component 103 (compliant, semi-compliant, or non-compliant) can determine the necessary number of cycles to clean the inflatable component 103. In some examples, the amount of contrast in the inflatable component 103 can determine the number of cycles necessary to clean the inflatable component 103.

The system 100 can be operable to detect when the inflatable component 103 is free from contrast inside the inflatable component 103 (e.g., the inflatable component 103 is clean and safe to use in a patient). In some examples, a sensor 115 can be placed directly upstream of the contamination sink 114. In some examples, the sensor 115 can include a chemical sensor. The sensor 115 can be any sensor(s) operable to detect iodine or other chemical components of the contrast to be cleaned out of the inflatable component 103. During every contamination removal cycle, the sensor 115 can measure the iodine (or other chosen chemical level) of the contaminated fluid. The system 100 can determine that the inflatable components 103 are free from contrast when the measured iodine level is below a threshold. In some examples, the threshold can be a concentration of about 0% iodine, about 0.5% iodine, about 1% iodine, about 1.5% iodine, about 2% iodine, about 2.5% iodine, about 3% iodine, about 3.5% iodine, about 4% iodine, about 4.5% iodine, or about 5% iodine. In another aspect, the threshold can be a concentration of about 0% iodine to about 1% iodine, about 1% iodine to about 2% iodine, about 2% iodine to about 3% iodine, about 3% iodine to about 4% iodine, or about 4% iodine to about 5% iodine.

In some examples, the system 100 can also be operable to determine if the inflatable component 103 has a leak. In some examples, the pressure transducer 125 can be configured to determine if there is a leak in the inflatable component 103 during the fluid fill stage (e.g., when fluid is supplied to the inflatable component 103). Once the cleaning solution fill stage begins, the controller can be configured to start a timer. The timer can be set to a desired fill stage time limit. The time limit can be about 1 minute to about 2 minutes, about 2 minutes to about 3 minutes, about 3 minutes to about 4 minutes, about 4 minutes to about 5 minutes, about 5 minutes to about 6 minutes, about 6 minutes to about 7 minutes, about 7 minutes to about 8 minutes, about 8 minutes to about 9 minutes, about 9 minutes to about 10 minutes, or more. If the inflatable component 103 does not reach the desired pressure during the time limit, the system 100 can be configured to provide an alarm that the inflatable component 103 is not filling to the desired pressure within the time limit, thereby indicating a potential leak or hole in the inflatable component 103. It will be appreciated that when the other fill sensors 120 described herein are used, the controller can run a timer for the inflatable component 103 to reach a desired parameter (e.g., weight, force, diameter increase, etc.) and determine that the inflatable component 103 has a leak when the desired parameter is not reached in the time limit.

In some examples, the pressure transducer 125 can be configured to determine if there is a leak in the inflatable component 103 after the cleaning process is completed (e.g., after all cycles are completed). After all the contaminated fluid has been pulled out of the inflatable component 103 after the last cycle, the open/close valve 118 can be placed in the closed state and the first pump 112 can provide a pressure to the inflatable component 103 to inflate the inflatable component 103 to a desired testing pressure. In some examples, the desired testing pressure can be the rated burst pressure of the inflatable component 103, an operating pressure of the inflatable component 103, or another pressure. Once the inflatable component 103 reaches the desired testing pressure, as measured by the pressure transducer 125, the pump can be turned off. The pressure transducer 125 can then be monitored for a testing time period to determine if the inflatable component 103 loses any pressure. If the pressure of the inflatable component 103 drops by more than a threshold pressure drop, the controller can provide an alarm indicating that the inflatable component 103 has a potential leak.

In some examples, while the inflatable component 103 is being filled by the at least one pressure source 112 (e.g., first pump 112), the system 100 can monitor the fill rate of the inflatable component 103 and determine if there is a leak or hole in the inflatable component 103 depending on the fill rate. In some examples, the fill rate can be determined as a rate of pressure increase. If the inflatable component 103 is not filling in accordance with the rate of pressure increase, the controller can produce an alert indicating the inflatable component 103 has a hole or a leak. Additionally, if the first pump 112 is a hydraulic piston, the fill rate can be determined by the displacement of the piston.

In some examples, the pressure transducer 125 can include an analog pressure gauge. In some examples, the pressure transducer 125 can include a digital pressure gauge. In some examples, the pressure transducer 125 can include a computer-controller pressure transducer to provide high accuracy in measuring the rates of pressure increase or decrease which can be characterized for more advanced leak recognition. In some examples, the pressure transducer 125 can include a pressure sensor. In some examples, the pressure transducer 125 can be used in conjunction with other fill sensors 120 described herein. In some examples, the fill sensors 120 described herein can be used as an alternative to the pressure transducer 125.

In some examples, multiple inflatable components 103 can be cleaned at one time, as illustrated in FIG. 3. The at least one medical device attachment port 102 can include a plurality of medical device attachment ports. Each medical device attachment port 102 can be operable to clean a single inflatable component 103. Each medical device attachment port 102 can be connected to the one or more fluidic paths 122 via the first manifold 104 and the second manifold 106. The first manifold 104 can have the same number of ports as the number of medical device attachment ports 102. The second manifold 106 can have the same number of ports as the number of medical device attachment ports 102. In some examples, the system 100 can have 1, 2, 3, 4, 5, 6, 7, 8, 9, 10, 11, 12, 13, 14, 15, 16, 17, 18, 19, or 20 medical device attachment ports 102. In some examples, the system 100 can have about 2 medical device attachment ports 102 to about 10 medical device attachment ports 102, about 10 medical device attachment ports 102 to about 20 medical device attachment ports 102, about 20 medical device attachment ports 102 to about 30 medical device attachment ports 102, about 30 medical device attachment ports 102 to about 40 medical device attachment ports 102, about 40 medical device attachment ports 102 to about 50 medical device attachment ports 102, about 50 medical device attachment ports 102 to about 60 medical device attachment ports 102, about 60 medical device attachment ports 102 to about 70 medical device attachment ports 102, about 70 medical device attachment ports 102 to about 80 medical device attachment ports 102, about 80 medical device attachment ports 102 to about 90 medical device attachment ports 102, or about 90 medical device attachment ports 102 to about 100 medical device attachment ports 102. It will be appreciated that multiple inflatable components 103 can also be attached to the system 100 illustrated in FIGS. 1-2 by adding additional medical device attachment ports 102.

In some examples, the medical device attachment ports 102 can be arranged in parallel. In some examples, every inflatable component 103 can be cleaned at the same time. Each medical device attachment port 102 can have the inlet port 107, the first outlet, and the second outlet in an open state at the same time. The open/close valve 118 can be placed in the closed state. The pressure transducer 125 can determine when all of the inflatable components 103 have reached the desired pressure. As illustrated in FIG. 3, the inflatable components 103 can all be in parallel and therefore the pressure value can be easily obtained. Once the inflatable components 103 reach the desired pressure, the first pump 112 can be turned off.

In some examples, each inflatable component 103 can be cleaned and/or tested sequentially. A first inflatable component 103 can be cleaned first by providing the fluid (e.g., cleaning solution to a first medical device attachment port 102 (e.g., at least one medical device attachment port 102)). The first medical device attachment port 102 can have the inlet port 107 in the open state, the first outlet port 109 in the open state, and the second outlet in the open state. The open/close valve 118 can be placed in the closed state. The inflatable component 103 can be provided with cleaning solution until the pressure transducer 125 measures that the inflatable component 103 has reached the desired pressure. The additional medical device attachment ports 102 can have the first outlet port 109 (e.g., the outlet connected to the inflatable component 103) in the closed state while the first inflatable component 103 is filled with fluid (e.g., cleaning solution). Once the first inflatable component 103 reaches the desired pressure, the inlet port 107, first outlet port 109, and second outlet can be placed in a closed state while the fluid (e.g., cleaning solution) sits in the first inflatable component 103 during the dwell period. The first outlet port 109 of a second medical device attachment port (e.g., at least one medical device attachment port 102) can then be placed in the open state and the second inflatable component 103 can be provided cleaning solution until the second inflatable component 103 reaches the desired pressure. In some examples, the fluid in the first inflatable component 103 can be removed using the contamination sink and the vacuum pressure. Then the inlet port 107, first outlet, and second outlet of the first medical device attachment port 102 (e.g., cleaning valve) can be closed. The inlet port 107, first outlet, and second outlet of the second cleaning valve can then be opened while the additional cleaning valves remained closed, and the process can be repeated. It will be appreciated that a similar method can be conducted for testing leaks in multiple inflatable component 103.

In some examples, multiple inflatable components 103 can be tested for leaks using the pressure transducer 125. When multiple inflatable components 103 are filled at the same time, the pressure transducer 125 can determine that one of the inflatable components 103 has a leak if the inflatable components 103 do not reach the desired pressure in the time limit, as described above. In some examples, each balloon can be connected to its own pressure transducer and open/close valve. For example, a pressure transducer and open/close valve can be located before the second manifold 106 and connected to each inflatable component 103 individually. The system 100 can then operate in substantially the same way as described above with respect to each individual inflatable component 103.

In some examples, the first outlet port 109 of the at least one medical device attachment port 102 and the inflatable component 103 can be contained in a closed environment. The closed environment can be connected to a pressure source operable to provide pressure (e.g., pressure to the external side of the inflatable component 103) to the closed environment. A pressure can be applied to the closed environment when the contaminated fluid is being removed from the inflatable component 103 to compress the inflatable component 103 and expel the contents of the inflatable component 103. By providing a positive pressure to the inflatable components 103, complete removal of the contaminated fluid can be ensured.

In some examples, the system 100 can be configured to clean a variety of different medical devices 101 with inflatable components 103. In some examples, the system 100 can be configured to clean over-the-wire (OTW) or rapid-exchange (RX; monorail) PTA balloon catheters. In some examples, the system 100 can be configured to clean scoring balloon catheters. The system 100 can be configured to clean atherectomy balloons. The atherectomy balloons can be configured to deliver RF energy to aid in breaking up calcified regions. In some examples, the system 100 can be configured to clean stent deliver PTA balloons. In examples, the system 100 can be configured to clean drug coated balloons. In some examples, the system 100 can be configured to clean hydrophilic coated balloons. In some examples, the medical device 101s with inflatable components 103 can have compliant, semi-compliant, and/or non-compliant inflatable components 103. All types of inflatable components can be cleaned by the system 100. The pressure provided to the medical device 101 via the at least one pressure source 112 (e.g., first pump 112) can depend on the type of inflatable component 103.

As illustrated in FIGS. 1 and 3, the system 100 can be a closed loop system. In some examples, the system 100 can be self-contained, such that external pressures do not affect the functioning of the system 100.

In some examples, as described herein, the system 100 for cleaning medical devices 101 with inflatable components 103 can be a pressure based device, as described above. Other exemplary systems using different parameters to determine desired cleaning and testing are described herein that do not require a pressure transducer or can be used in conjunction with a pressure transducer. In some examples, the measurement devices can work in combination with each other, the system 100 can utilize flow and pressure sensors, or force gauges, pressure sensors, and scales. It will be appreciated that the components described herein can be combined in different orders or variations.

A flow rate based detection method can be used as an alternative, or in conjunction with, the pressure based detection method described herein. In some examples, the fill sensor 120 can be a flow rate sensor 117. A flow rate sensor 117 can be located upstream of the at least one medical device attachment port 102. The flow sensor can measure flow rates of fluid to the inflatable component(s) 103 and determine a volume of fluid provided to the inflatable component(s) 103 based on the flow rate and amount of time the cleaning solution has been provided to the inflatable component(s) 103. A flow rate into the lumen (described in further detail below) and the inflatable component 103 can be measured. When the volumes inside the inflatable component 103 and the guidewire lumen reach a desired volume, determined based on the flow rate and the time the flow rate is provided, the system 100 can provide a vacuum pressure to remove the contaminated cleaning solution from the lumen and the inflatable component 103.

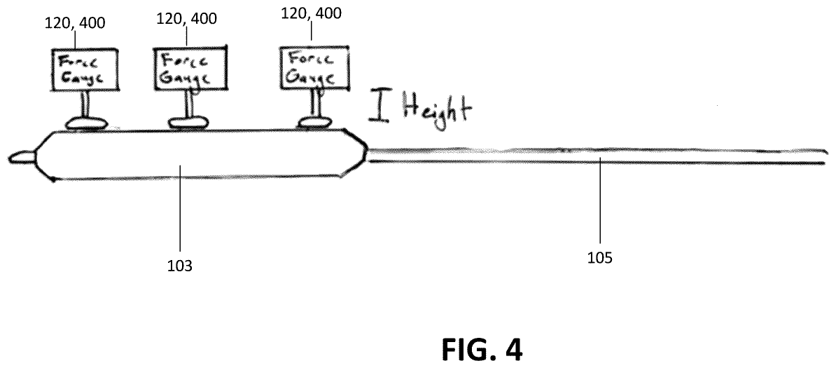

Another detection method to ensure full surface area contact of the fluid (e.g., cleaning solution) with the inflatable component 103 can include a force gauge system. A force gauge can be used to determine the fluid (e.g., cleaning solution) has come into contact with the full surface of the inflatable component 103 by measuring a force from the exterior of an inflated inflatable component 103. In some examples, the fill sensor can include one or more force gauge sensors 400. As illustrated in FIG. 4, one or more force gauges 400 can be located on a side of the inflatable component 103. The one or more force gauges 400 can measure a force of expansion of the inflatable component 103 from a proximal to a distal end of the inflatable component 103. The height (e.g., distance from unexpanded inflatable component 103 surface to force gauge 400) of the one or more force gauges 400 can be determined based on the type of inflatable component 103 to be cleaned. A compliant inflatable component 103 can have a greater height of the one or more force gauges 400 than a non-compliant inflatable component 103. The inflatable component 103 can be inflated until the amount of force measured by the force gauges indicates that the inflatable component 103 has been sufficiently expanded for the entire interior surface area of the inflatable component 103 to be in contact with the fluid (e.g., cleaning solution). The one or more force gauges 400 can also be used to determine if there is a leak in the inflatable component 103. When no force is measured in the force gauge after a period of time in the cleaning process, it can be determined that there is a leak in the inflatable component 103. Furthermore, similar to the pressure transducer measurement described herein, the inflatable component 103 can be inflated with fluid, the pump can be turned off, the open/close valve can remain in the closed state, and the one or more force gauges 400 can be monitored for any drop in force. If there is a drop in force, the controller can trigger an alarm that an inflatable component 103 has a potential leak.

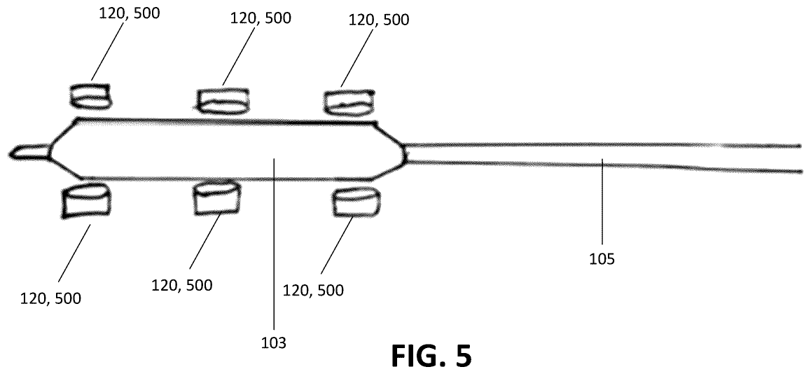

Another detection method to ensure full surface area contact of the fluid (e.g., cleaning solution) with the inflatable component 103 can involve a proximity sensor based system. In some examples, the fill sensor 120 can include one or more proximity sensors 500. One or more proximity sensors 500 can be placed around the inflatable component 103 being pressurized, as illustrated in FIG. 5. The one or more proximity sensors 500 can measure a distance between the wall of the inflatable component 103 and the one or more proximity sensors 500. The inflatable component 103 shaft sits at a fixed distance from the one or more proximity sensors 500. The one or more proximity sensors 500 can be configured such that when a desired distance to the inflatable component 103 wall is measured, the inflatable component 103 is fully inflated. Similarly, the one or more proximity sensors 500 can be used to determine if there is a leak or hole in the inflatable component. After a set period of time, if the inflatable component 103 wall does not reach the desired distance to the one or more proximity sensors 500, it can be determined that the inflatable component 103 has a hole or a leak. The set period of time can be calculated by determining the amount of time it will take based on the pressure rate provided by the pressure pump to reach the desired pressure inside the inflatable component 103 for full contact of the interior surface area of the inflatable component 103 by the fluid. The set period of time can be the amount of time it will take to reach the desired pressure at the provided pressure rate, or 1.5 times the amount of time it will take to reach the desired pressure at the provided pressure rate, or 2 times the amount of time it will take to reach the desired pressure at the provided pressure rate. Further, when pressure is locked in the system, the inflatable component 103 walls should remain at a constant distance from the one or more proximity sensors 500. If the inflatable component 103 walls begin moving away from the one or more proximity sensors 500 while the pressure in the system is locked, the system can determine that the inflatable component 103 has a leak.

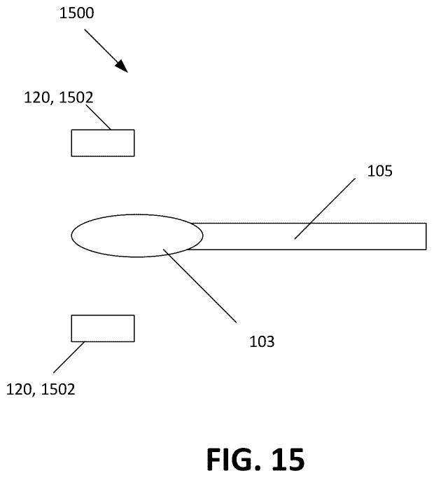

Another detection method to ensure full surface area contact of the fluid (e.g., cleaning solution) with the inflatable component 103 can involve a machine vision system. The fill sensor can include one or more cameras 1502. As illustrated in FIG. 15, the machine vision system 1500 can have one or more cameras 1502 operable to observe the inflatable component 103. The one or more cameras 1502 can be configured to determine whether the inflatable components 103 are fully expanded based on a known expansion diameter (e.g., set expansion diameter based on the necessary diameter to ensure full contact of the fluid with the entire interior surface area of the inflatable component 103) of the inflatable component 103 to be cleaned. The machine vision system 1500 can also determine if there is a leak if complete expansion is not achieved in the set period of time, as described above. Additionally, the machine vision system 1500 can be used to determine if there is a hole or leak in the inflatable component 103 by observing the inflatable components 103 when pressure is locked in the device. If the inflatable components 103 begin deflating when the pressure is locked in the device, the machine vision system 1500 can determine that the inflatable component 103 has a leak.

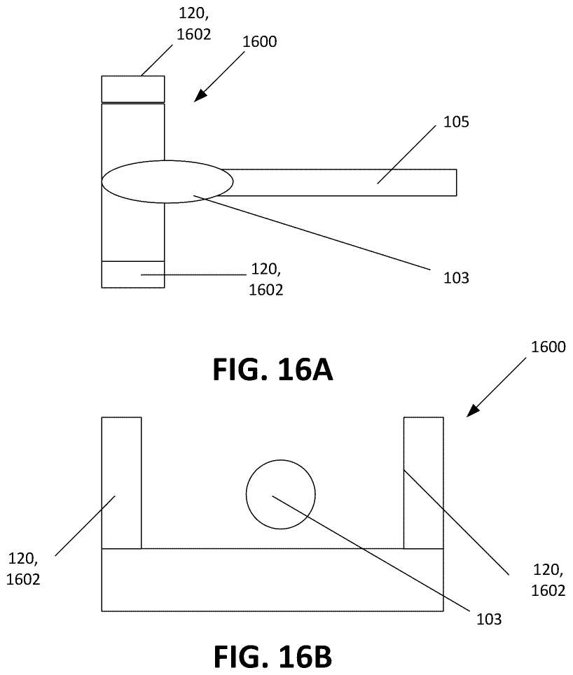

Another detection method to ensure full surface area contact of the fluid (e.g., cleaning solution) with the inflatable component 103 can involve a laser micrometer system. As illustrated in FIGS. 16A-16B, the laser micrometer system 1600 can include one or more lasers 1602. The fill sensor 120 can include one or more lasers 1602. A laser micrometer 1600 can use light to accurately measure the diameters of the inflatable components 103 when fully expanded (e.g., the expansion diameter necessary to ensure complete contact of the fluid with the interior surface area of the inflatable component 103) or deflated. Multiple diameter readings of the medical device 101 with an inflatable component 103 (e.g., proximal, distal, and middle sections of the inflatable component 103) yields the inflatable component 103 profile when fully inflated. The dimensions can be characterized by the type of the inflatable component 103 being cleaned. When full expansion to the expansion diameter of the inflatable component 103 is achieved, the fluid (e.g., cleaning solution) is in contact with the full surface area of the inflatable component 103. Additionally, when pressure is locked in the system 100, the laser micrometer 1600 can determine if the inflatable component 103 has a leak by determining a change in the diameter.

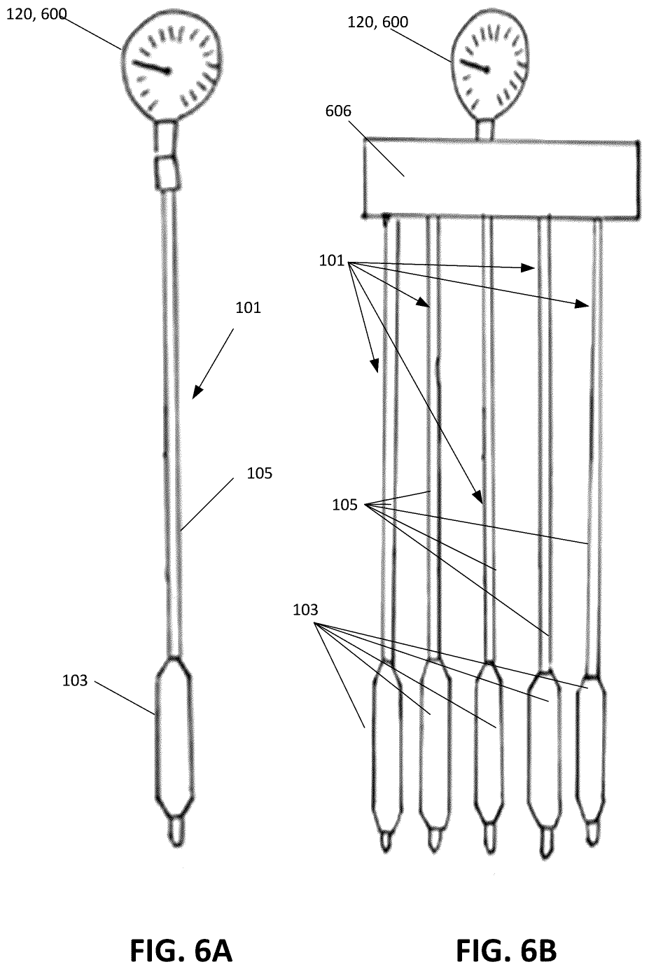

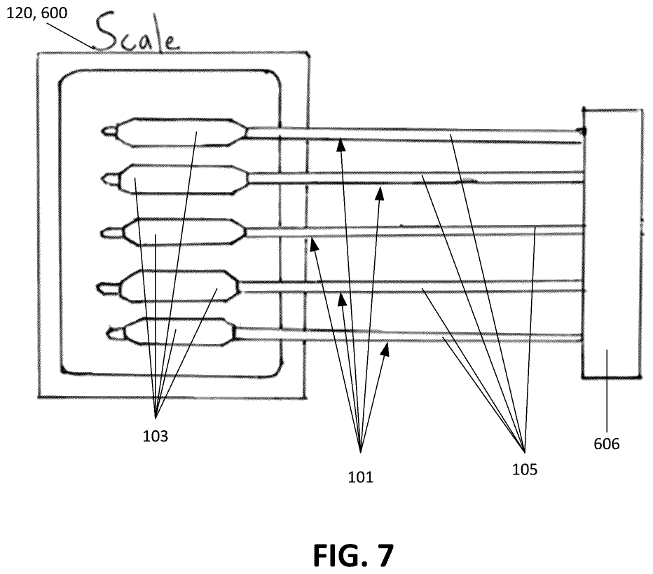

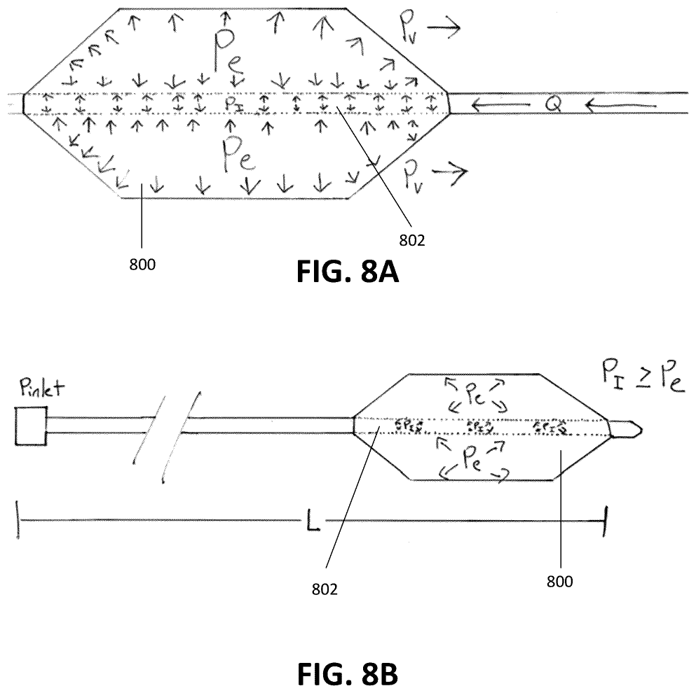

Another detection method to ensure full surface area contact of the fluid (e.g., cleaning solution) with the inflatable component 103 can include a weight based system, as illustrated in FIGS. 6A, 6B, and 7. The fill sensor 120 can include one or more scales 600. First, the volume of an expanded inflatable component 103 is determined. The volume of the expanded inflatable component 103 can be multiplied by the density of the fluid (e.g., cleaning solution) to determine a weight of the fluid (e.g., cleaning solution) when the inflatable component 103 is fully expanded. The weight of the medical device 101 with an inflatable component 103 can be added to the weight of the cleaning solution necessary for full expansion of the inflatable component 103. The medical device 101 with an inflatable component 103 can be connected to a scale 600 via an inflation lumen 121 housed within a catheter body 105. When the scale 600 reaches the weight of the inflatable component 103 plus the fluid (e.g., cleaning solution) at full expansion, the inflatable component 103 is determined to be fully expanded. The weight based system can also detect holes or leaks in the inflatable component 103 if the weight does not reach a fully expanded weight in a period of time. Further, the weight system can determine that the inflatable component 103 has a hole or a leak by locking the pressure in the system and determining if the weight changes over time.

As illustrated in FIGS. 6A-6B, the weight system can be a vertical system that hangs the inflatable components 103 vertically. If multiple inflatable components 103 are to be cleaned at one time, an individual scale 600 for each inflatable component 103 can be used, as illustrated in FIG. 6A, or a single scale 600, as illustrated in FIG. 6B, for multiple inflatable components 103 can be used. As illustrated in FIG. 6B, when a single scale 600 is used, each inflation lumen 121 (housed within catheter body 105) can be connected to a manifold 606. If a single scale 600 for multiple inflatable components 103 is used, the combined expansion weight of the inflatable components 103 will be used as the determinant of full expansion of all inflatable components 103. As illustrated in FIG. 7, the weight system can be a horizontal system that places the inflatable components 103 on a scale 600 while attached horizontally to a manifold 606 (e.g., via the inflation lumen 121 contained within the catheter body 105). Multiple inflatable components 103 of the same type or a different type can be cleaned and tested by the weight based system by adding the weights of the inflatable components 103 together.

Further provided herein is a system for cleaning one or more lumens 123 (e.g., aspiration lumen, therapeutic delivery lumen, guidewire lumen, or any lumen besides the inflation lumen 121) of the medical device 101 with an inflatable component 103. The lumens 123 can be disposed within (e.g., contained within) the catheter body 105. For example, the medical device 101 may have an inflation lumen 121 which delivers the fluid to the inflatable component 103 as well as multiple other lumens 123 (e.g., guidewire lumen, aspiration lumen, therapeutic delivery lumen, etc.). The system for cleaning the one or more lumens 123 can be substantially the same as the systems described herein for cleaning the inflatable component 103. A highly important factor in cleaning the medical device 101 with an inflatable component 103 is maintaining sufficient pressure in the one or more lumens (e.g., lumens other than the inflation lumen 121) so that the one or more lumens 123 do not collapse due to external forces on the one or more lumens created by inflating the inflatable component 103. If the external forces on the one or more lumens 123 are not counteracted, the one or more lumens will collapse, thereby destroying the medical device 101 with an inflatable component 103. If the one or more lumens 123 are not to be cleaned, a guidewire or other solid material can be inserted in the one or more lumens to counteract the forces exerted on the one or more lumens 123 by the pressure necessary to inflate the inflatable component 103. Additionally, a similar problem occurs during removal of the fluid from the inflatable component 103 using a vacuum pressure. In some examples, the system for cleaning the one or more lumens 123 can be integrated with the system 100.

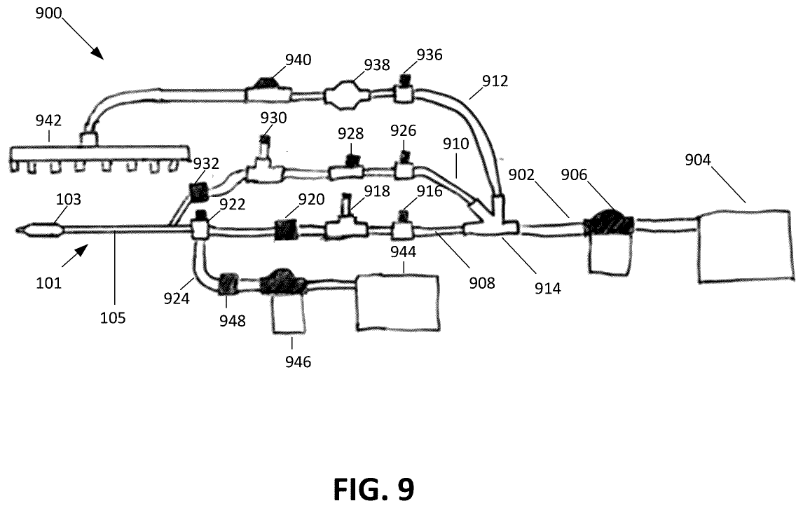

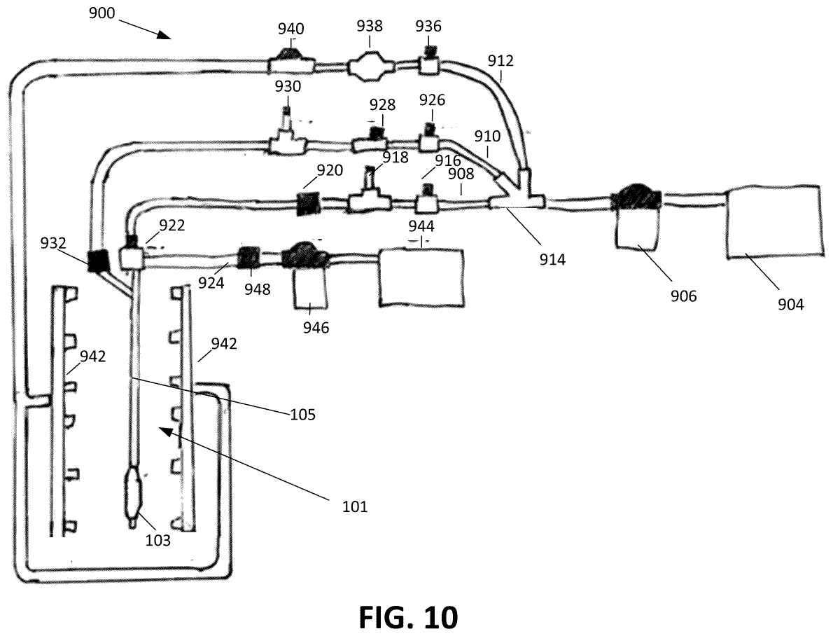

As illustrated in FIGS. 8A-8B, the pressure provided to the inflatable component 800 also exerts a pressure on the one or more lumens 802. In some examples, the system for cleaning the one or more lumens can use the Hagen Poiseuille, Eq. 2, principle to create enough pressure in the one or more lumens 802 to counteract the exterior pressure from pressurizing the inflatable component 800.

$$Δ p = \frac{8 μ L Q}{π {R}^{4}} \qquad ( Eq . 2 )$$

Where ΔP is the pressure difference between the two ends (e.g., the inlet pressure and the pressure necessary at Pᵢ (i.e., the internal pressure of the lumen)), μ is the dynamic viscosity of the cleaning solution, L is the length of the lumen, R is the radius of the lumen, and Q is the volumetric flow rate. Using the Hagan Poiseuille principle can provide the desired pressure needed from the at least one pump for pressurizing the lumen 802. The Hagan Poiseuille principle can be rearranged as shown in Eq. 3.

$$Q = \frac{Δ P ( π {R}^{4} )}{8 μ L} \qquad ( Eq . 3 )$$

ΔP can initially be Pᵢₙₗₑₜ-Pₑ, where Pᵢₙₗₑₜ is the inlet pressure and Pₑ is the pressure inside the inflatable component 800. Pₑ can be substituted for Pᵢ where Pᵢ is the pressure inside the lumen. Using the known radius of the inflatable component 800, known length of medical device where Pᵢ needs to be greater than or equal to Pₑ, and the viscosity of the fluid (e.g., cleaning solution), the necessary volumetric flow rate (Q) can be calculated and provided to the lumen 802 to counteract the pressure exerted by the inflatable component 800 on the lumen 802 (e.g., guidewire lumen, therapeutic delivery lumen, aspiration lumen, etc.). In some examples, the volumetric flow rate (Q) can be monitored and provided by a flow regulating valve located upstream from the lumen 802 and downstream from the pump.

In some examples, a pressure transducer can monitor P_{I} and Pₑ and open/close valves can be actuated to maintain a P_{I} that is greater than or equal to Pₑ. In some examples, other methods of maintaining Pᵢ equal to or above Pₑ can be used.

In some examples, the lumen cleaning system can be configured to determine that the one or more lumens are free of contrast in substantially the same way as the inflatable component cleaning device provided herein. In some examples, the one or more lumens can be determined to be free of contrast using the systems and methods described in U.S. Publication No. 2023/0152135.

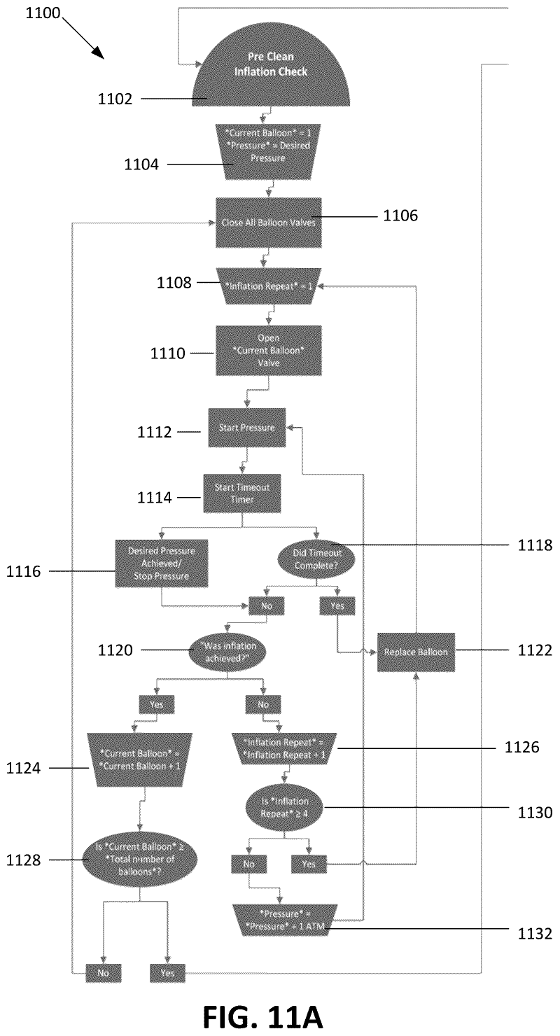

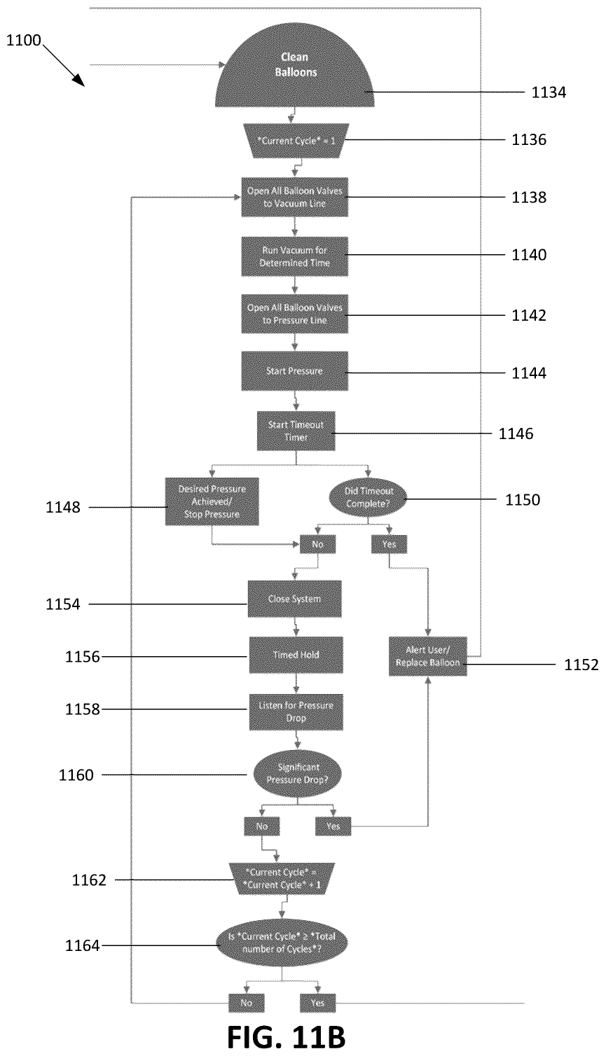

Further provided herein is a system for cleaning an exterior of the medical device. As illustrated in FIGS. 9-10, the system for cleaning the exterior of the medical device can have a spraying manifold 942, at least one pump 906, and a cleaning solution source 904. The spraying manifold 942 can be fluidically connected to the cleaning solution source 904 and the at least one pump 906. When the inflatable component 103 of the medical device 101 is connected to the at least one medical device attachment port 922, the spraying manifold 942 can be configured to spray the exterior of the medical device 101 with the cleaning solution. In some examples, the spraying manifold 942 can spray the exterior of the medical device 101 while the inflatable component 103 is being cleaned by the system 100, 900. In some examples, the exterior cleaning system can further include a flow meter 940, a flow regulating valve 938, and an open/close valve 936 for providing a necessary flow rate for a desired time to clean the exterior of the inflatable component 103. In some examples, the system for cleaning the exterior of the medical device can be integrated with the system 100.

In another examples, the spraying manifold 942 can be fluidically connected to a water source. The spraying manifold 942 can be provided water from the water source and a pressure from the at least one pressure source 906. The spraying manifold 942 can be used to rinse the cleaning solution off the medical device. In some examples, the system can have multiple spraying manifolds 942 operable to spray the exterior of the medical device 101 with a cleaning solution and/or water.

In some examples, the exterior cleaning system can be a cleaning solution sink. The medical device 101 with an inflatable component 103 can sit in the cleaning solution sink when the lumen 121, 123 and the inflatable component 103 are cleaned, thereby cleaning the exterior of the medical device 101 with an inflatable component 103 at the same time. When the lumen 121, 123 and the inflatable component 103 are determined to be free of contrast, the medical device 101 with an inflatable component 103 can be removed from the cleaning solution sink.

In some examples, the spray manifold 942 can begin providing fluid to the exterior of the medical device while the fluid is being vacuumed from the lumen 121, 123 and the inflatable component 103. In some examples, the pressure provided by the fluid on the exterior of the inflatable component 103 can speed up the contaminated fluid removal process by adding additional pressure to expel the fluid in the inflatable component 103.

In some examples, the inflatable component 103, lumen 121, 123, and exterior of the medical device 101 can be cleaned at the same time. In some examples, the system for cleaning the inflatable component, the system for cleaning the lumen, and the system for cleaning the exterior can all have different connections and components (e.g., separate devices). In some examples, the system for cleaning the inflatable component, the lumen, and the exterior can be incorporated in a single system.

In some examples, the medical devices 101 can be connected to the system 100, 900 horizontally. The medical devices 101 can then be placed in a sink, such that the medical devices 101 can be soaked or sprayed on the exterior, while the inflatable component 103 and lumen 121, 123 are cleaned using the systems described herein. In some examples, the exterior cleaning and lumen cleaning of the medical device 101 can be performed using the same pump source that is used to pressurize and inflate the inflatable components 103.

In some examples, the medical devices 101 can be hung vertically from the system 100, 900. The medical devices 101 can be placed in a container, which can allow more medical devices 101 to be cleaned at once while occupying less space. Additionally, hanging the medical devices 101 vertically can put less stress on the shaft and connectors/hubs of the medical devices as they are inflated and deflated. The container can be connected to the system for cleaning the exterior of a medical device with an inflatable component. The container can allow the exterior of the medical devices 101 to be cleaned while the lumens and inflatable components of the medical devices 101 are cleaned. In some examples, using a plumbing and pump configuration, the exterior cleaning and interior cleaning of the medical device 101 can be performed using the same pressure source or multiple pressure sources.

In some examples, the system 100, 900 can be completely automated. The system 100, 900 can have one or more controllers in communication with the pumps, pressure transducers, cleaning valves, open/close valves, contamination valve, pressure supply valve, and fluid supply valve. The one or more controllers can be operable to turn on the pumps and open or close the various ports of the cleaning valves, open/close valves, contamination valve, pressure supply valve, and the fluid supply valve. The controller can further be operable to calculate various parameters (e.g., time, inflatable component pressure, rate of pressure increase or decrease, pressures drops in the inflatable components 103, flow of fluid in the lumen, flow of fluid in the exterior, and other parameters as described herein) of the system to determine which valves to open or close. The controller can also be in communication with the pressure transducer and provide data related to the safety (e.g., leak or no leak) of the inflatable components 103. The controller can be able to receive a pressure value from the pressure transducer and open or close different valves to move to a different step in the cleaning process. A detailed cleaning process for a controller is described further herein.

FIGS. 9-10 illustrate a system for cleaning a medical device 101 with an inflatable component 103. As illustrated in FIGS. 9-10, the system 900 can be completely interconnected (e.g., one pump controls the inflatable component 103, lumen (within the catheter body 105), and exterior cleaning (exterior of medical device 101)). The system 900 can include a first fluidic path 902, a cleaning solution source 904, and a first pressure source 906 (e.g., any pump or pressure source described herein). The first fluidic path 902 can provide a fluid (e.g., cleaning solution) from the cleaning solution source 904 to an inflatable component fluidic path 908, a lumen fluidic path 910, and an exterior fluidic path 912. In some examples, the first fluidic path 902 can be fluidically connected to the inflatable component fluidic path 908, the lumen fluidic path 910, and the exterior fluidic path 912 via a 4-way fitting 914. The 4-way fitting can divide the first fluidic path 902 into the inflatable component fluidic path 908, the lumen fluidic path 910, and the exterior fluidic path 912. In some examples, other configurations of the fluidic paths can be used such that the fluid from the cleaning solution source 904 is provided to the inflatable component 103, one or more lumens, and exterior of the medical device with the inflatable component 103.

In FIGS. 9-10, the fluid can flow as described herein. The first pressure source 906 can be turned on and pull fluid from the cleaning solution source 904. The fluid can flow through the first fluidic path 902 to a distribution fitting (e.g., 4-way fitting 914). At the 4-way fitting 914, the first fluidic path 902 splits into an inflatable component fluidic path 908, a lumen fluidic path 910, and an exterior fluidic path 912. The fluid can flow through all the paths simultaneously or be controlled to flow in one or more of the paths. The fluid can flow through the inflatable component fluidic path 908 through the open/close valve 916, the pressure transducer 918, the first manifold 920, and into the at least one medical device attachment port 922 (e.g., inflatable component cleaning valve, diverter valve, two-way valve, three-way valve, or any other attachment port described herein). The fluid can be pulled from the inflatable component 103 by turning on the second pressure source 946 (e.g., pump operable to provide vacuum pressure), thereby pulling the fluid from the inflatable component 103 through the at least one medical device attachment port 922, the second manifold 948, and into the contamination sink 944. The second pressure source 946 can be any of the pumps or pressure sources described herein.

The fluid can flow through the lumen fluidic path 910 through the open/close valve 926, the pressure regulator valve 928, the pressure transducer 930, the lumen manifold 932 and into the one or more lumens of the medical device. The fluid can flow through the exterior fluidic path 912 through the open/close valve 936, the flow regulating valve 938, and the flow meter 940 to the spraying manifold 942. The spraying manifold 942 can provide cleaning solution to the exterior of the medical device.

The inflatable component fluidic path 908 can include an open/close valve 916 downstream from the 4-way fitting, a pressure transducer 918 downstream from the open/close valve 916, a first manifold 920 downstream from the pressure transducer 918, and at least one medical device attachment port 922 downstream from the first manifold 920. A inflatable component 103 can be connected to the at least one medical device attachment port 922. The at least one medical device attachment port 922 can have an inlet for receiving fluid from the cleaning solution source 904, via the inflatable component fluidic path 908 and the 4-way fitting 914. The at least one medical device attachment port 922 can have a first outlet for attaching to the inflatable component 103 and a second outlet for attaching to a contamination fluidic path 924.

The lumen fluidic path 910 can include an open/close valve 926 downstream from the 4-way fitting 914, a pressure regulator valve 928 downstream from the open/close valve, a pressure transducer 930 downstream of the pressure regulator valve 928, and a lumen manifold 932 downstream of the pressure transducer 930. The lumen manifold can have an inlet for receiving fluid from the cleaning solution source 904 via the 4-way fitting 914 and an outlet for attaching to the lumen of the medical device with an inflatable component 103. The lumen manifold 932 can allow for multiple lumens to be attached to the system 900. The fluid can travel from the cleaning solution source 904 through the lumen fluid path 910 to the one or more lumens of the medical device. The fluid can then flow through the lumen of the medical device and out into a second contamination sink.

The exterior fluidic path 912 can include an open/close valve 936 downstream from the 4-way fitting 914, a flow regulating valve 938 downstream from the open/close valve 936, a flow meter 940 downstream from the flow regulating valve 938, and at least one spray manifold 942 downstream from the flow meter 940. The spray manifold 942 can be operable to spray an exterior of the medical device with an inflatable component 103. In another aspect, as illustrated in FIG. 10, the system 900 can include two spraying manifolds 942.

The contamination fluidic path 924 can include an outlet manifold 948 connected to the second outlet of the inflatable component cleaning valve and a second pressure source 946, and a contamination sink 944.

The system 900 can be operable to function in at least three phases, a cleaning phase, a removal phase, and a testing phase. During the cleaning phase, the inflatable component 103, lumen 121, 123, and exterior of the medical device 101 with the inflatable component 103 can be cleaned. The first pressure source 906 can pull fluid from the cleaning solution source 904 and provide the fluid to the inflatable component fluidic path 908, the lumen fluidic path 910, and the exterior fluidic path 912. The open/close valves 916, 926, 936 can all be placed in the open state and fluid can flow to the inflatable component 103, the lumen 121, 123, and the exterior of the medical device 101 with an inflatable component 103. The second outlet of the at least one medical device attachment port 922 can be placed in the closed state to ensure that pressure build up inside the inflatable component 103 occurs. The pressure transducer 918 of the inflatable component fluidic path 908 can monitor the pressure inside of the inflatable component. Once the pressure inside the inflatable component 103 reaches a desired pressure, the first pressure source 906 can be turned off and the fluid can be allowed to dwell in the inflatable component for a dwell period. During the dwell period, the open/close valves 916, 926, 936 can be placed in the closed state.

During the cleaning phase, the pressure regulator valve 928 and the pressure transducer 930 of the lumen fluidic path can monitor and adjust the pressure inside the one or more lumens. The pressure inside the one or more lumens can be maintained as described herein. Further, by supplying fluid to the one or more lumens at the same time as the inflatable component 103, the lumen 121, 123 can also be cleaned.

Additionally, during the cleaning phase, the exterior of the medical device with an inflatable component 103 can be sprayed by the spraying manifold 942, thereby cleaning the exterior of the inflatable component 103 and/or medical device. The flow regulating valve 938 can maintain a desired flow rate on the exterior of the medical device with an inflatable component 103 such that the medical device with an inflatable component 103 is cleaned but the pressure created by the flow of the exterior fluid does not counteract the pressure being provided inside the inflatable component. The flow meter 940 can monitor the flow of the exterior fluid to ensure that the correct flow rate of exterior fluid is being provided.

After the dwell period described herein, the removal phase can begin. The removal phase can be operable to remove the fluid from the inflatable component 103 and the lumen 121, 123. At the removal phase, the second outlet of the inflatable component cleaning valve can be opened such that fluid can be pulled from the inflatable component 103 to the contamination fluidic path 924. During the removal phase, the second pressure source 946 is turned on. The second pressure source 946 can provide a vacuum pressure to the inflatable component 103 to remove the fluid to the contamination fluidic path 924. The fluid (e.g., contaminated/dirty fluid) from the inflatable component 103 can be removed to the contamination sink via the second outlet of the at least one medical device attachment port 922. In some examples, a calculated time of removal can be implemented, and the second pressure pump can run for a calculated time. In some examples, a fluid sensor can be placed in the contamination fluidic path 924. When the fluid sensor determines the second pressure source 946 is no longer pulling fluid from the inflation lumen 121 and the inflatable component 103, the second pressure source 946 can be turned off. Additionally, the chemical sensor 115 described herein can be placed along the contamination fluidic path 924 to determine if the inflatable component 103 is free of contrast. In some examples, the exterior cleaning can take place during the removal stage rather than during the cleaning stage, thereby providing pressure on the inflatable component 103 to more quickly expel the fluid (e.g., contaminated fluid) from the inflatable component 103.

In some examples, the exterior cleaning can take place during the removal phase. In this aspect, the exterior fluidic path 912 is closed off during the cleaning phase by closing the open/close valve 936 of the exterior fluidic path 912. When the removal phase begins, the open/close valves 916, 926 of the inflatable component fluidic path 908 and the lumen fluidic path 910 can be closed and the open/close valve 936 of the exterior fluidic path can be opened. The first pressure source 906 and the cleaning solution source 904 can provide cleaning solution to only the exterior fluidic path 912 during the removal phase. When fluid is provided to the exterior during the removal phase (e.g., the outside of the medical device is cleaned while fluid is removed from the lumen 121, 123 and the inflatable component 103) the removal of the fluid from the lumen 121, 123 and the inflatable component 103 can be sped up by the additional exterior pressure.

After completion of the removal phase, the cleaning phase can be run again. The cleaning phase and removal phase can be run until it is determined that the inflatable component 103 and the one or more lumens are free of contrast below a predetermined threshold.

After the inflatable component 103 and the one or more lumens 121, 123 are determined to be free of contrast, the testing phase can begin. The testing phase can consist of the steps of the cleaning phase up to the dwell period. During the dwell period, the open close valves 916, 926, 936 and the second outlets of the at least one medical device attachment port 922 can be placed in the closed state. The pressure transducers 918, 930, of the inflatable component fluidic path 908 and the lumen fluidic path 910, can monitor the pressure inside the inflatable component 103 and the lumen to determine if there are any leaks. If there is a pressure drop exceeding a threshold pressure drop, it can be determined that the inflatable component 103 or the lumen 121, 123 has a leak. In some examples, each inflatable component 103 and lumen 121, 123 can be tested for leaks individually or at the same time as described herein.

It will be appreciated that the fill sensors 120 discussed herein can be used in conjunction with or as alternatives to the pressure transducers described for the system 900. Furthermore, the system 900 can include any of the components in any combination or order as discussed herein. The system 900 can also be configured to clean multiple medical devices 101 with inflatable components 103 at once, as described herein, by utilizing the manifolds 920, 932, and 948 and multiple medical device attachment ports.

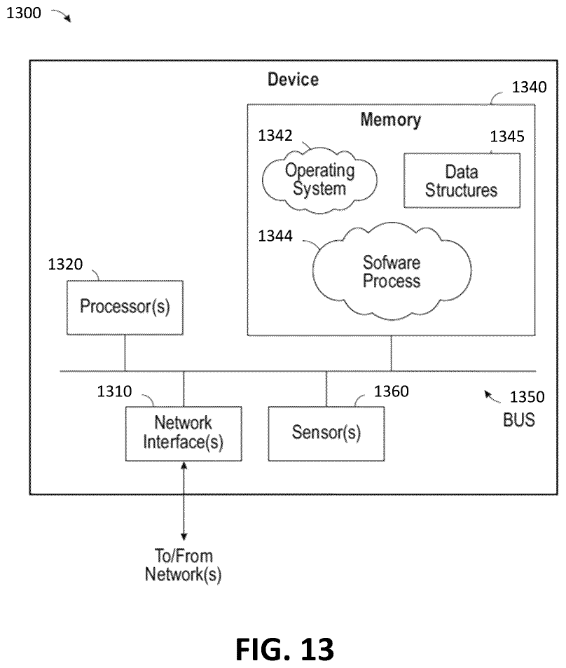

FIG. 13 is a block diagram of an exemplary controller 1300. Controller 1300 is configured to perform processing of data and communicate with the various fill sensors, valves, ports, and other components described herein. In operation, controller 1300 communicates with one or more of the above-discussed components and may also be configured to communication with remote devices/systems.

As shown, controller 1300 includes hardware and software components such as network interfaces 1310, at least one processor 1320, sensors 1360 (e.g., sensors for determining position of components, power delivered to motors, etc.) and a memory 1340 interconnected by a system bus 1350. Network interface(s) 1310 can include mechanical, electrical, and signaling circuitry for communicating data over communication links, which may include wired or wireless communication links. Network interfaces 1310 are configured to transmit and/or receive data using a variety of different communication protocols.

Processor 1320 represents a digital signal processor (e.g., a microprocessor, a microcontroller, or a fixed-logic processor, etc.) configured to execute instructions or logic to perform tasks for operation of the systems 100, 900. Processor 1320 may include a general purpose processor, special-purpose processor (where software instructions are incorporated into the processor), a state machine, application specific integrated circuit (ASIC), a programmable gate array (PGA), an individual component, a distributed group of processors, and the like. Processor 1320 typically operates in conjunction with shared or dedicated hardware, including but not limited to, hardware capable of executing software and hardware. For example, processor 1320 may include elements or logic adapted to execute software programs and manipulate data structures 1345, which may reside in memory 1340.

Fill sensors 120, which may include sensors for determining a fill level (e.g., contact level) of the inflatable components disclosed herein, typically operate in conjunction with processor 1320 to perform measurements, and can include special-purpose processors, detectors, transmitters, receivers, and the like. In this fashion, fill sensors may include hardware/software for generating, transmitting, receiving, detection, logging, and/or sampling various parameters of the systems 100, 900.

Memory 1340 comprises a plurality of storage locations that are addressable by processor 1320 for storing software programs and data structures 1345 associated with the embodiments described herein. An operating system 1342, portions of which may be typically resident in memory 1340 and executed by processor 1320, functionally organizes the device by, inter alia, invoking operations in support of software processes and/or services 1344 executing on controller 1300. These software processes and/or services 1344 may perform processing of data and communication with controller 1300, as described herein. Note that while process/service 1344 is shown in centralized memory 1340, some examples provide for these processes/services to be operated in a distributed computing network.

It will be apparent to those skilled in the art that other processor and memory types, including various computer-readable media, may be used to store and execute program instructions pertaining to functions of the systems 100, 900 described herein. Also, while the description illustrates various processes, it is expressly contemplated that various processes may be embodied as modules having portions of the process/service 1344 encoded thereon. In this fashion, the program modules may be encoded in one or more tangible computer readable storage media for execution, such as with fixed logic or programmable logic (e.g., software/computer instructions executed by a processor, and any processor may be a programmable processor, programmable digital logic such as field programmable gate arrays or an ASIC that comprises fixed digital logic. In general, any process logic may be embodied in processor 1320 or computer readable medium encoded with instructions for execution by processor 1320 that, when executed by the processor 1320, are operable to cause the processor 1320 to perform the functions described herein.

Further provided herein is a method for cleaning a medical device having a inflatable component. The method can be configured to clean a variety of medical devices having inflatable components. The method can be carried out using the systems described herein. In some examples, the method can be carried out using other devices or systems operable to carry out the steps of the method. In some examples, the methods can be automated using a computer.

The system for cleaning the medical device can be primed and purged automatically before a medical with an inflatable component is attached to the system. The system can be primed and purged by closing the cleaning valves and providing fluid to the fluidic path or fluidic paths, depending on the device of system to be used for cleaning.

The method of cleaning the medical device with an inflatable component can vary depending on the type of inflatable component attached to the medical device. Using an inflatable component compliance assessment (e.g., Eq. 1) to determine the correct cleaning method can be used for semi-compliant and compliant inflatable components.

$$Compliance Assessment = \frac{OD @ RBP - OD @ NP}{OD @ RBP} \times 100 \% \qquad ( Eq . 1 )$$

Using Eq. 1, the outer diameter of the inflatable component can be measured and compared to the labeled diameter of the configuration to determine if the internal surface area of the medical device with an inflatable component is being expanded greater than the expansion threshold (e.g., full expansion) by the cleaning solution. The nominal pressure necessary for full expansion, and therefore full contact of the fluid with the entire interior surface area of the inflatable component can be ensured.

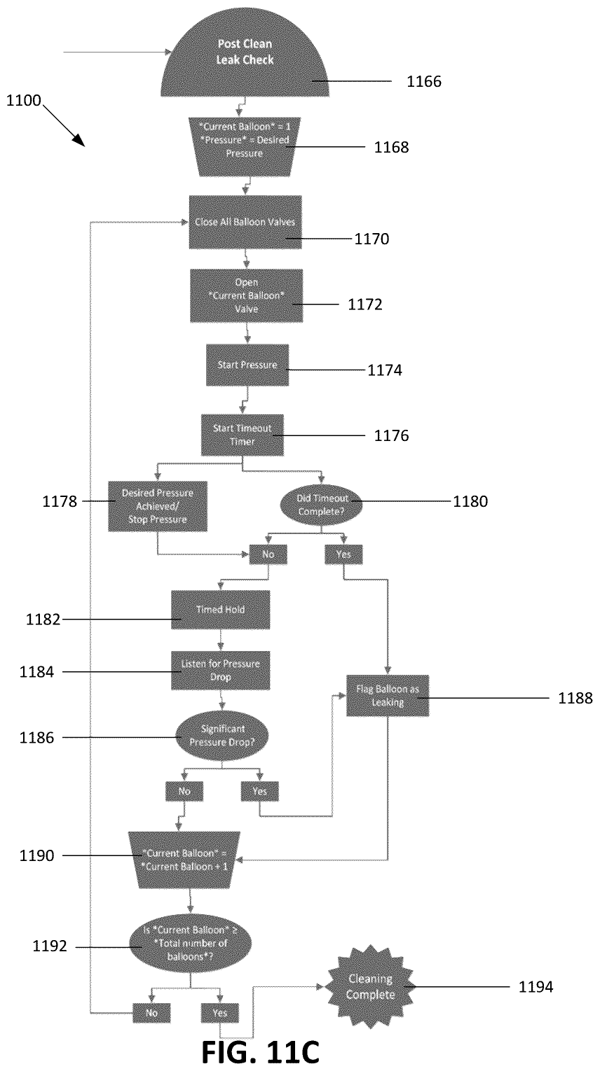

FIG. 11A is a first part of the method 1100 which continues on FIG. 11B and then continues further on FIG. 11C. The method 1100 of FIGS. 11A-11C can be conducted using a controller (e.g., can be automated). The method 1100 can begin by connecting one or more inflatable components to the system or device and selecting a desired recipe depending on the type of the inflatable component (e.g., compliant, semi-compliant, non-compliant). The selected recipe can include a desired pressure for cleaning, a dwell period, and other parameters described herein (e.g., lumen pressure, flow rate of exterior cleaning fluid, etc.).

At step 1102, the method 1100 can include running a pre-clean inflation check. At step 1104, the inflation component to be checked can be selected (e.g., if more than one inflation component is to be cleaned then a pre-clean inflation check can be conducted for each inflation component).

At step 1106, all balloon valves (e.g., valves operable to provide or prevent pressure from entering the inflation component) can be closed. At step 1108, a counter can count the number of pre-use inflation checks run on each inflation component. At step 1110, the current inflation component (e.g., current balloon valve to provide pressure to the inflation component) can be opened. Opening the inflation component valve in step 1110 can include opening the inlet and outlets of at least one medical device attachment port (e.g., the inlet is open and the first outlet is open). Depending on the system or device to be used, an open/close valve or the second outlet of the at least one medical device attachment port can be placed in a closed state. The valve to be closed should lock pressure in the system or device such that the inflatable component begins inflating.

At step 1112, the at least one pressure source pump to pressurize the inflatable component can be turned on (e.g., pressure is supplied to the current inflation component to be checked). At step 1114, a timer can begin. At step 1116, the at least one pressure source has achieved a desired or stop pressure and if timeout did not complete at step 1118, the method 1100 moves to step 1120. If timeout completes at step 1118, the inflatable component likely has a leak, and the inflatable component is replaced at step 1122.

At step 1120, whether inflation of the inflatable component was achieved is determined (e.g., desired pressure reached, desired weight reached, desired diameter change reached, or any other measurement for determining inflation of the inflation component as described herein). If inflation is achieved at step 1120, the method 1100 proceeds to step 1124. If inflation is not achieved at step 1120, the method proceeds to step 1126. At step 1124, the current inflation component is set to the next inflation component and the method moves to step 1128. At step 1128, the method determines whether there are any additional inflation components to be checked or if all inflation components have been checked. If all inflation components have been checked, the method 1100 proceeds to step 1134. If there are additional inflation components to check, the method 1100 begins at step 1102 for the next inflation component to check.

If inflation is not achieved at step 1120, the method 1100 proceeds to step 1126. At step 1126, the method 1100 sets the inflation check number of the current balloon to the next inflation check number (e.g., 1 to 2, 2 to 3, 3 to 4, etc.). At step 1130, the method 1100 determines whether the next inflation check number is greater than a set maximum inflation check number (e.g., 3 maximum inflation checks for a single inflation component, 4 maximum inflation checks for a single inflation component, 5 maximum inflation checks for a single inflation component, etc.). At step 1130, if the next inflation check number exceeds the maximum inflation check number, the method 1100 goes to step 1122 and the inflation component is replaced. At step 1130, if the next inflation check number is less than the maximum inflation check number, the method 1100 proceeds to step 1132. At step 1132, the system is depressurized and the method 1100 proceeds to step 1102 to run another inflation check on the current inflation component.

If multiple inflatable components are to be cleaned at one time, the pre-clean inflation check is run for each inflatable component individually by opening only the valves required to inflate the inflatable component to be checked and closing the rest of the valves. If every inflatable component passes the pre-clean inflation check, the method 1100 can move on to step 1134. If one or more inflatable components fail the inflation check, the inflation check is run at least 2 more times on the failing inflatable components. If an inflatable component fails all of the pre clean inflation checks, the inflatable component is deemed as having a leak or the system has a failure. An operator can then inspect the inflatable component to determine if there is a leak and check the system to determine if there is an error. As illustrated in FIGS. 11A-C, the inflation check for a failing inflatable component can be run 4 times.

At step 1134, the cleaning process can begin. The cleaning process can begin at a first cycle. At step 1136, the current cycle can be determined by the method 1100. At step 1138, a cycle can start with opening all of the valves to the contamination fluidic path (to the contamination sink). At step 1140, the method 1100 can include running the vacuum pump (the first pump, the second pump, venturi pump, or any other pump operable to vacuum out the system) for a desired time to clean the system of any fluid and/or pressure from the pre-clean inflation check. At step 1142, after desired vacuum time has completed, the valves (e.g., inlet and first outlet of the medical device attachment ports) can be opened such that the inflatable components can be pressurized (e.g., the inlets and first outlets of the at least one medical device attachment ports are placed in the open state and the open/close valves or second outlets of the medical device attachment ports are placed in the closed state, depending on the device or system to be used). At step 1144, the at least one pressure source can then be turned on and fluid (e.g., from the cleaning source sink) can be provided to the one or more inflatable components. At step 1146, a timer can begin. At step 1148, the desired pressure from the at least one pressure source is reached. At step 1150, it is determined whether the timeout for the timer has completed (e.g., whether pressure was reached within the time limit). At step 1150, if the desired pressure was reached before the timeout period ends, the method 1100 moves to step 1152. If the desired pressure is not reached in the timeout period, the method moves to step 1152, where a user is alerted and the method 1100 returns to step 1102. In some examples, the desired pressure can be the determined by the pressure transducer. At step 1154, the system can be closed (e.g., the valves are closed such that the fluid remains within the inflation component and the pressure transducer can monitor the pressure in the inflation component). At step 1156, a dwell period can be observed. The dwell period can be any of the dwell periods described herein. In some examples, a force (e.g., via a rocker plate or other motion mechanism) can be applied to the inflation component to move the fluid within the inflation component. At step 1158, the pressure transduce can monitor the pressure within the inflation component. At step 1160, the method 1100 can determine whether the pressure drop exceeds a pressure drop threshold. If the pressure drop exceeds a pressure drop threshold, the method 1100 can move to step 1152 where a user is alerted and the method 1100 goes back to step 1102. If the pressure is maintained in the inflation component at step 1160, the method 1100 can continue to step 1162. During the dwell period, the pressure transducer can monitor the pressure in the inflatable components for any drop in pressure, if the pressure drop exceeds a threshold, an operator can be alerted and the operator can begin the pre clean inflation check again. If the inflatable components do not reach the desired pressure within the time period set by the timer, the operator can be alerted and the operator can begin the pre clean inflation check again.

At 1162, the method 1100 determines a number of a next cycle and moves to step 1164. At step 1164, the method 1100 determines if a next cycle is needed or if the next cycle is greater than the desired number of cycles. If a next cycle is needed, the method 1100 repeats from step 1134. If a next cycle is not needed, the method 1100 moves to step 1166. The cleaning process can have 1, 2, 3, 4, 5, 6, 7, 8, 9, 10, 11, 12, 13, 14, 15, 16, 17, 18, 19, or 20 cycles.

After all of the cycles of the cleaning process have successfully been completed, the method 1100 can include beginning an individual leak testing process at step 1166. At step 1168, the inflation component and corresponding desired pressure can be selected. As illustrated in FIG. 11C, the individual leak testing process (Post Clean Leak Check) can include closing all of the medical device attachment ports, at step 1170. At step 1172, a first selected medical device attachment port can be opened. At step 1174, the at least one pressure source can be turned on to provide pressure to the first inflatable component to be leak checked. At step 1176, a timer can begin when the at least one pressure source is turned on. The timer can set a time period for the inflatable component to reach the desired pressure. At step 1178, the method 1100 can determine whether the desired pressure is reached. At step 1180, the method 1100 can determine whether the desired pressure was reached within before the end of the timeout period set by the timer. At step 1182, If the inflatable component reaches the desired pressure, as determined by the pressure transducer during the time period, the required valves (i.e., the open/close valve or the second outlet of the medical device attachment port depending on the system or device used) can be closed to lock pressure inside the inflatable component. If the pressure does not reach the desired pressure during the time period set by the timer, the inflatable component can be flagged as leaking at step 1188. When the desired pressure is reached in the timeout period set by the timer, the pressure source pump can be turned off. At step 1182, the pressure can be held in the inflatable component for another time period. At step 1184, the pressure transducer can monitor the pressure in the inflatable component for a pressure drop. At step 1186, If the inflatable component has a pressure drop exceeding the pressure threshold, the inflatable component can be flagged as having a potential leak (step 1188). At step 1186, if the inflatable component does experience a pressure drop exceeding the pressure threshold, the method 1100 can move to step 1190. At step 1190, a number of the next inflation component to test can be determined. At step 1192, the method 1100 can determine whether the number of the next inflation component to test is greater than the total number of inflation components to check. If the number of the next inflation component to test is greater than the total number of inflation components to check, the method 1100 can be completed at step 1194. If the number of the next inflation component to check is less than or equal to the number of inflation components to check, the method can continue to step 1166 to check the next inflation component. The process can be repeated to check every inflatable component that was cleaned during the cleaning process if multiple inflatable components were cleaned at one time. After all the inflatable components have been checked for leaks, the inflatable components have been reprocessed and the medical devices with the inflatable components are ready for medical use.

In further aspects, the method 1100 can include cleaning the exterior of the medical device. The exterior of the medical device can be cleaned while the inflatable component is being cleaned, after the inflatable component has been cleaned, or before the inflatable component is cleaned. The exterior of the medical device can be cleaned by spraying the exterior with a cleaning solution using at least one spraying manifold. some examples, the exterior of the medical device can be cleaned by soaking the medical device in a cleaning solution. In some examples, the at least one spraying manifold can be in fluid communication with a cleaning solution source (e.g., the same cleaning solution source as the cleaning solution source for the inflatable component or a different cleaning solution source). In some examples, the at least one spraying manifold can be provided a pressure by the at least one pressure source pump. In some examples, the at least one spraying manifold can be provided a pressure by a different pressure source pump. In some examples, the exterior can be cleaned during the vacuum (e.g., fluid removal stage) of the cleaning process, thereby providing an external pressure to the inflatable component to expel the fluid more quickly.

In some examples, the method 1100 can further include cleaning one or more lumens of the medical device. In some examples, the one or more lumens of the medical device can be cleaned while the inflatable component and the exterior are cleaned, before the inflatable component and the exterior are cleaned, or after the inflatable component and the exterior are cleaned. In some examples, cleaning the one or more lumens of the medical device can include connecting the one or more lumens (e.g., guidewire lumen, therapeutic delivery lumen, aspiration lumen, etc.) to the lumen manifold, providing a cleaning solution to the lumen through the lumen manifold via a second cleaning solution source in fluid communication with the lumen manifold and allowing the cleaning solution to flow out of the lumen into a contamination sink.

If the lumen cleaning is conducted during the inflatable component cleaning process, the one or more lumens of each medical device with an inflatable component can be provided a pressure sufficient to counteract the force exerted by the pressure in the inflatable component on the lumen, as described herein. If cleaning of the lumen does not occur during cleaning of the inflatable component, a guidewire or other solid material can be placed in the one or more lumens to ensure that the one or more lumens do not collapse due to the pressure in the inflatable component exerted on the one or more lumens.

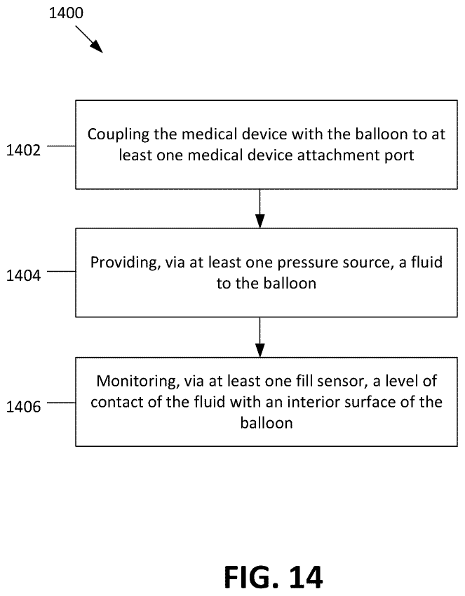

FIG. 14 illustrates a method 1400 for cleaning a medical device with an inflatable component. The method 1400 can be conducted using any of the devices, systems, or methods described herein. For example, method 1400 can utilize one or more steps in method 1100 and/or utilize system 100, 900.

At block 1402, the method 1400 can begin by coupling the medical device with the inflatable component to at least one medical device attachment port. As described herein, the medical device attachment port can include a cleaning valve, diverter valve, three-way valve, two-way valve, or any other type of attachment port. In some examples, a plurality of medical devices with inflatable components can be coupled to a plurality of medical device attachment ports. The at least one medical device attachment port can be in fluid communication with one or more fluidic paths. In some examples, the at least one medical device attachment port can be in fluid communication with an inflatable component fluidic path of the one or more fluidic paths. In some examples, the one or more fluidic paths can be in fluid communication with a cleaning solution source containing a fluid (e.g., cleaning solution). In some examples, the inflation lumen of the medical device can be coupled to the at least one medical device attachment port. The inflation lumen can be in fluid communication with the inflatable component.

At block 1404, the method 1400 can include providing, via at least one pressure source, a fluid to the inflatable component. In some examples, the at least one pressure source can be any of the pumps or pressure sources described herein. The at least one pressure source can be in fluid communication with the one or more fluidic paths. The at least one pressure source can be in fluid communication with the cleaning solution source. Providing the fluid to the inflatable component can include providing a pressure to the cleaning solution source to move (e.g., direct) the fluid from the cleaning solution source to the at least one medical device attachment port through the one or more fluidic paths (e.g., inflatable component fluidic path). The at least one medical device attachment port can be configured such that the fluid flows through the at least one medical device attachment port, into the inflation lumen, and then into the inflatable component. In some examples, the at least one medical device attachment port can have one or more inlets and outlets (e.g., diverter valve) that are operable to be actuated between open and closed states. The one or more inlets and outlets can be actuated such that fluid moves into the inflatable component, fluid moves out of the inflatable component, fluid is prevented from entering the inflatable component, and/or fluid is prevented from exiting the inflatable component.

At block 1406, the method 1400 can include monitoring, via at least one fill sensor, a level of contact of the fluid with an interior surface of the inflatable component. The at least one fill sensor can be any of the fill sensors described herein (e.g., one or more pressure transducers, force gauges, scales, proximity sensors, machine vision systems, and/or laser micrometers). As described herein, the at least one fill sensor can be operable to determine when the fluid has contacted the entire interior surface of the inflatable component based on a measured parameter (e.g., weight, force, diameter, pressure, etc.).

The method 1400 can further include observing a dwell period once the level of contact of the fluid indicates that the interior surface of the inflatable component is entirely contacted with the fluid. In some examples, observing the dwell period includes turning off the at least one pressure source. In some examples, observing the dwell period includes locking the fluid in the inflatable component by actuating the at least one medical device attachment port such that fluid cannot exit the inflatable component. In some examples, the dwell period can be a sufficient period of time for the fluid (e.g., cleaning solution) to break down any contrast, dirt, or material within the inflatable component. In some examples, the dwell period can be about 30 seconds to about 1 minute, about 1 minute to about 5 minutes, or about 5 minutes to about 10 minutes.

The method 1400 can further include determining whether the inflatable component has a leak based on a rate of change in the level of contact of the fluid with the interior surface of the inflatable component. In some examples, a leak can be determined in the dwell period. While all the fluid is prevented from escaping the inflatable component via the at least one medical device attachment port, the at least one fill sensor can detect any changes in the parameters of the inflatable component (e.g., pressure, weight, force, diameter, etc.). If the parameter changes above a threshold amount or a rate of change exceeds a threshold, it can be determined that the inflatable component has a leak. In some examples, determining whether the inflatable component has a leak can include monitoring the parameter of the inflatable component with the fill sensor while the fluid is being provided to the inflatable component. For example, if the parameter (e.g., pressure, weight, force, diameter, etc.) is not changing as expected, it can be determined that the inflatable component likely has a leak.

The method 1400 can further include removing, via the at least one pressure source, the fluid from the inflatable component. In some examples, the at least one pressure source can include a single pressure source. The single pressure source can provide a positive pressure to fill the inflatable component and a negative pressure (e.g., vacuum pressure) to remove the fluid from the inflatable component via the one or more fluidic paths (e.g., inflatable component fluidic path). In some examples, the at least one pressure source can include a first pressure source for filling the inflatable component and a second pressure source for removing the fluid from the inflatable component. In some examples, the fluid removed from the inflatable component can be directed, via the at least one pressure source, to a contamination sink. In some examples, a chemical sensor can be in fluid communication with the contamination sink. For example, the chemical sensor can be disposed within the contamination sink. In some examples, the chemical sensor can be disposed along the one or more fluidic paths (e.g., contamination fluidic path) between the at least one medical device attachment port and the contamination sink. The chemical sensor can be operable to determine whether the inflatable component has been cleaned of contrast. For example, the chemical sensor can be configured to measure an iodine concentration of the fluid removed from the inflatable component. If the iodine concentration is below a threshold, as described herein, it can be determined that the inflatable component is cleaned of contrast.

The method 1400 can be repeated 1, 2, 3, 4, 5, 6, 7, 8, 9, 10, 11, 12, 13, 14, 15, 16, 17, 18, 19, 20, or more times until the inflatable component is sufficiently cleaned of contrast.

In some examples, the method 1400 can further include cleaning one or more lumens of the medical device. The one or more lumens can include any lumen of the medical device other than the inflation lumen. For example, the one or more lumens can include an aspiration lumen, a therapeutic delivery lumen, a guidewire lumen, and any other type of lumen. In some examples, the one or more lumens are provided fluid via the cleaning solution source by the at least one pressure source. For example, the one or more lumens can be connected to one lumen fluidic path of the one or more fluidic paths. The one or more lumen fluidic paths can include a lumen fluidic path for each lumen to be cleaned.

In some examples, the fluid flows through the one or more lumens and exits the one or more lumens into a contamination container. In some examples, the one or more lumens are plugged such that the fluid remains in the one or more lumens and is pulled out by a vacuum pressure from the at least one pressure source. In some examples, the one or more lumens are cleaned (e.g., provided fluid) at the same time as the inflatable component is provided fluid such that the pressure counteracts a pressure from providing fluid to the inflatable component (e.g., inflating the inflatable component). In some examples, when the one or more lumens are not provided fluid at the same time as the inflatable component, the one or more lumens can be filled with a solid material or guidewire such that the one or more lumens do not collapse under the pressure from the inflating inflatable component and pressure in the inflation lumen.

In some examples, the method 1400 can further include cleaning an exterior surface of the medical device. In some examples, the medical device can be submerged in a container filled with cleaning solution. In some examples, one or more sprayers can be configured to spray a fluid (e.g., cleaning solution) on to the exterior of the medical device. For example, the one or more sprayers can be in fluid communication with an exterior fluidic path of the one or more fluidic paths. The at least one pressure source can be configured to provide fluid from the cleaning solution source, through the exterior fluidic path, to the one or more sprayers. In some examples, the exterior of the medical device is cleaned at the same time as the inflatable component and/or one or more lumens. In some examples, the exterior of the medical device is cleaned while fluid is being removed from the inflatable components and the one or more lumens. When the exterior of the medical device is cleaned as fluid is being removed from the inflatable component and one or more lumens, the additional pressure provided by the fluid sprayed on the exterior of the inflatable component can push the fluid out of the inflatable component faster, thereby expediting the fluid removal process.

It will be appreciated that any of the devices and systems can be used in the methods disclosed herein. The different inflatable component fill and leak detection systems and devices can be used instead of, or in conjunction with, the pressure transducers described in the method. It will also be appreciated that different components of the devices, systems, and methods described herein can be used in conjunction with each other, some components can be used and some components may not be used in certain devices, systems, and methods of the present disclosure.

### EXAMPLES

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes could be made in the disclosure and still obtain a like or similar result without departing from the spirit and scope of the disclosure, therefore all matter set forth is to be interpreted as illustrative and not in a limiting sense.

### Example 1

Single-use Percutaneous Transluminal Angioplasty (PTA) devices are catheters that have an inflatable component attached to the distal end of the catheter and can be pressurized using a contrast/saline mixture. These devices are used all over the vasculature of the human body. Therefore, increasing the associated risk to the patient when using these devices. Currently, there are no known medical device reprocessing methods that have undertaken the task to reprocess PTA devices due to their high-risk profiles. The inflatable components are made of various polymers and materials that allow them to withstand high pressures, and some lower pressures. Nonetheless, these inflatable components can be fragile if not handled correctly. This makes the reprocessing strategy more difficult.

A device for washing and cleaning single use catheters with an inflatable component mounted on the distal end of the catheter (PTA balloons, Drug Coated Balloons, Balloon Expandable Stents, etc.).

### Devices to be Reprocessed

### Plain Old Balloon Angioplasty

Percutaneous Transluminal Angioplasty (PTA) dilatation balloon catheters are used in PTA procedures to treat obstructive lesions of native or synthetic arteriovenous dialysis fistulae as well as post-dilatation of stents and stent-grafts in the peripheral vasculature.

Generally, PTA balloon catheters are either over-the-wire (OTW) or rapid-exchange (RX; monorail) in design. Both systems consist of a balloon inflation lumen and a guidewire lumen.

The inflation lumen extends from the inflation hub at the proximal end of the catheter through the full length of the catheter shaft to the balloon mounted at the distal end of the catheter. The guidewire lumen of OTW balloon catheters extends through the full length of the catheter, while the guidewire lumen in RX balloon catheters is limited to a select distal portion of the catheter. In either system, radiopaque markers within the balloon portion and/or along the shaft of the device can be present to facilitate fluoroscopic visualization of device position and orientation.

Clinically, PTA balloon catheters are introduced percutaneously through an introducer sheath at the vascular access site and advanced over the guidewire to the target lesion. The distal tip of the catheter is advanced through the stenosis, centering the balloon portion according to radiopaque markers. An inflation device is then used to apply inflation pressure sufficient to expand the balloon, restoring intraluminal diameter and improving blood flow. The balloon is then deflated and withdrawn.

### Scoring Balloons

There are many variations of balloon catheters that can have different indications for use but with a similar design as the plain old balloon angioplasty platform. There are scoring balloons that are meant to mechanically aid in breaking calcified plaque inside the vessels. These devices typically have scoring wires on the outside of the balloon and are inflated to pressure using contrast agent while allowing contact between the scoring wires and the calcified legion.

### Atherectomy Balloons-RF Energy Delivery (Lithotripsy)

In addition to scoring balloons, there are balloons that can deliver RF energy to aid in breaking up the calcified legions. These devices are typically connected to consoles that deliver the RF energy and create ultrasound pulses to aid in breaking calcified lesions. The balloons are also inflated to pressure using contrast agent to aid the physician visually during the procedure.

### Stent Delivery PTA Balloons

This type of catheter has a balloon at the distal end of the catheter that has a stent loaded onto the balloon. The catheter is advanced to the legion and inflated to pressure in order to deliver the stent. The balloon is pressurized to pressure using contrast media to aid in placement of the stent.

### Drug Coated Balloons

Drug Coated Balloons (DCB) are indicated to deliver drug to a calcified lesion to aid in treatment. The catheter has a balloon at the distal end of the catheter that is coated with a specified drug, and it is inflated to pressure using contrast media.

As detailed above, there are many variations of balloon catheters across the medical device industry that are intended for different purposes. However, there is one commonality between the different catheters; and that is that they are used with contrast solution to aid the physician in seeing placement of the balloon under fluoroscopy. The idea of this cleaning machine is that it will be able to efficiently clean the interior and exterior of these catheters without damaging the catheter and allowing it to be reprocessed without loss of integrity and performance over time.

### Hydrophilic Coated Balloons

Balloon catheters are coated with hydrophilic coatings that make it easier to track the catheter through tortuous anatomy inside the human body. Other than the functionality of tracking through anatomy, this balloon catheter functions like any other PTA device that is inflated using contrast solution when pressurizing.

### Compliant, Semi-Compliant, & Non-Compliant Balloon Catheters

Depending on the application of procedure, there are various kinds of balloon catheters that are made from different materials (polymers, etc.) that function in different manners. Balloons that are compliant are typically rated for lower pressures and conform to the shape of the lesion when inflated. As opposed to semi-compliant balloons that are typically stronger materials, semi-compliant balloons are able to dilate tougher lesions in the body while maintaining most of the original shape when inflated to the desired pressure. Lastly, non-compliant balloon catheters are typically high-pressure balloons without much compliance. Non-compliant balloons are very durable and will retain their shape when inflated to desired pressure under the lesion.

### Cleaning Process Flowchart

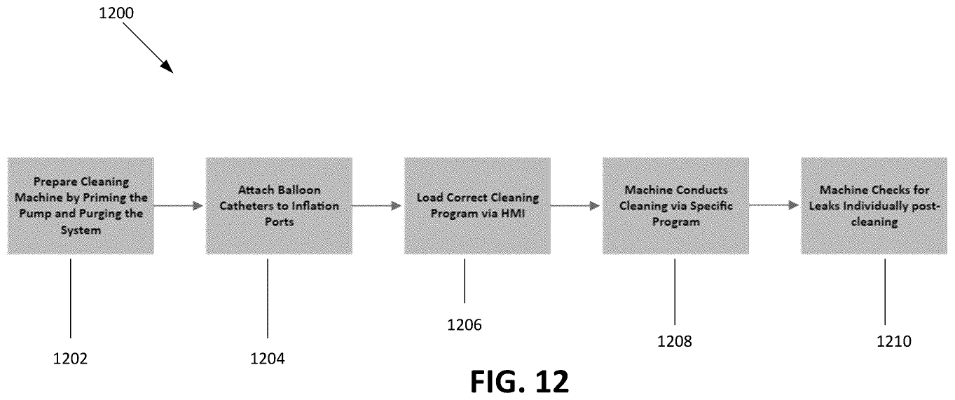

A cleaning process flow chart is illustrated in FIG. 12. The cleaning process can include preparing the cleaning machine/system by priming and purging the pump and purging the system, attaching a balloon catheter to an inflation port, loading a correct cleaning program via HMI, conducting the cleaning via the specific program, and checking the balloon for leaks individually post cleaning.

As illustrated in FIG. 12, the method 1200 for cleaning the medical devices with inflatable components can begin at block 1202. At block 1202, the method 1200 can include preparing the cleaning system by priming the pump and purging the system. At block 1204, the method 1200 can include attaching the inflatable component (e.g., via the inflation lumen) to the inflation ports (e.g., medical device attachment ports). At block 1206, the method 1200 can include loading a cleaning program for the system based on the type of inflatable component to be cleaned. At block 1208, the method 1200 can include conducting the cleaning process via the program. In some examples, conducting the cleaning process can be entirely automated by the cleaning system. At block 1210, the method 1200 can include checking the inflatable components for leaks. In some examples, checking the inflatable components for leaks can be entirely automated by the system.

Surface Area Contact for Cleaning Catheters with Inflatable Components

The cleaning system was developed on this concept that there needs to be full contact of cleaning agent with the internal surface area of the inflation lumens of the catheters. Inflatable component catheters can be pressurized to different pressures from nominal pressure all the way to rated burst pressure. However, the concept of building pressure is based on an enclosed system that builds pressure upon using different incompressible fluids, air, gases, etc.

Therefore, it is important for full contact of surface area be achieved using the cleaning agent. When pressure is built up, the inflatable component commences to expand until the system is fully enclosed and pressure is built up inside the catheter.

Compliant inflatable components, semi-compliant inflatable components, and non-compliant inflatable components behave very differently from each other. Therefore, the cleaning process can vary depending on the type of inflatable component catheter that is being cleaned. Using the Inflatable component Compliance Assessment to determine the correct cleaning method can be used for semi-compliant and compliant inflatable components.

$$Compliance Assessment = \frac{OD @ RBP - OD @ NP}{OD @ RBP} \times 100 \% \qquad ( Eq . 1 )$$

The compliance assessment in Eq. 1 measures the growth of the inflatable component at rated burst pressure and nominal pressure to assess the percentage of the inflatable component. Using this equation, the outer diameter of the inflatable component can be measured and compared to the labeled diameter of the configuration to determine if the internal surface area of the inflatable component catheter is being fully expanded by the cleaning solution.

Cleaning Process for Inflation Lumen via Machine

The cleaning machine is primed, and the system is purged automatically via a recipe created for the machine. Once primed and purged, inflatable component catheters (POBA, Stent Delivery Catheters, DCBs, etc.) are attached to the luer ports of the machine. A specific recipe is chosen by the operator that can actuate the cleaning process using a proprietary (contrast cleaning age). This program pressurizes/depressurizes the catheters with a cleaning age for multiple cycles until the inflation lumen of the device is free from contrast. Pressure is equal in parallel for equivalent sized pipes. Using a manifold to hold the catheters in parallel with each other allows the system to pressurize the entire manifold, and measurements of the pressure for all the inflatable components can be taken with a single transducer with confidence. Any number of inflatable components can be pressurized together in parallel if the pump is able to overcome the head loss. Lastly, the machine can have the capability to check for any leaks, pinholes, bursts, etc. by listening for drops in pressure. After the cleaning process to ensure no integral damage has been caused to the catheter post-cleaning.

### Cleaning Process of the Guidewire Lumen of the Catheter

The machine is attached to another pump that conducts the cleaning of the guidewire lumen of the device. The entire system functions together at the same time to conduct the cleaning of the guidewire lumen, inflation lumen, and exterior of the catheter.

Cleaning an inflatable component catheter is based on the volume of cleaning agent needed to make full contact with the internal surface area of the inflatable component and inflation lumen. Pressure does not build up unless full expansion of the inflatable component is created. Full expansion of the inflatable component indicates that the internal surface area is being contacted by the cleaning agent. Additionally, during the cleaning process the cleaning agent is vacuumed out of the inflatable component catheter. Inherently, pulling a vacuum creates a negative pressure (Pv as seen in FIG. 8A) inside the inflatable component and onto the inner membrane of the catheter, posing the same problem during inflation of the system.

Using the Hagen Poiseuille principle, the system creates enough pressure during cleaning of the guidewire lumen to counteract the exterior pressure from pressurizing the inflation lumen. During use of inflatable component catheters, it is always recommended to inflate while being over a guidewire. This is due to the pressure buildup inside the inflatable component that can cause physical damage to the inner membrane of the device (guidewire lumen). At times, this inner membrane can collapse during pressurization and deflation of the catheter if it is not supported by a guidewire. Therefore, the concept behind resolving this problem during cleaning utilizes the Eq. 4.

$$Δ p = \frac{8 μ LQ}{π {R}^{4}} \qquad ( Eq . 4 )$$

$$Δ p = pressure difference between the two ends$$

$$μ = dynamic viscosity$$

$$L = length of pipe$$

$$Q = volumetric flow rate$$

$$π = pi$$

$$R = pipe radius$$

FIG. 8A shows forces of pressure exerted during cleaning.

When pressurizing the inflatable component catheter during cleaning, a force of Pₑ is created. The force from Pₑ is exerted circumferentially in every direction as depicted in FIG. 8A. Consequently, the force from Pₑ is exerted onto the inner membrane of the catheter during this step. Therefore, an equal or greater force of P_{I} should be created to counteract this force during cleaning and provide the structural support for the inner membrane.

To create P_{I}, using the Hagen Poiseuille equation can be re-arranged to calculate the needed Volumetric Flowrate of Q as follows in Eq. 5.

$$Q = \frac{Δ P ( π {R}^{4} )}{8 μ L} \qquad ( Eq . 5 )$$

Q (Volumetric flowrate) is determined by the capability of equipment and can be a variable that can change depending on application of cleaning process. The critical parameter that stays true is that P_{I}≥P_{E}. Therefore, different design of experiments can be conducted using different volumetric flowrates to ensure to maintain P_{I}≥P_{E}.

Depending on the type of inflatable component catheter being reprocessed, the dimensions are known such as the Radius (inner membrane), fluid viscosity (depending on cleaning agent properties), and Length of catheter at the point where you need P_{I} to be greater than or equal to Pₑ (pressure loss is experienced based on length of pipe).

FIG. 8B shows a catheter demonstrating pressure exerted by inflatable component (Pₑ), pressure of the inner lumen at the outlet (P_{I}) and pressure at the inlet (Pᵢₙₗₑₜ).

Pₑ can then be substituted in for P_{I} in the Hagen Poiseuille equation as seen below in Eq. 6 and Eq. 7.

$${P}_{e} = {P}_{I} \qquad ( Eq . 6 )$$

$$Q = \frac{{P}_{inlet} - {P}_{e} ( π {R}^{4} )}{8 μ L} \qquad ( Eq . 7 )$$

As for the vacuum process, the same logic above can be applied. If enough Q is supplied into the guidewire lumen, the pressure exerted at P_{I} will be great enough the counteract the pressure created by the vacuum (P_{V})

### Detailed Components of Cleaning Machine

The system comprises any number of diverting valves, each fitted with a luer connector on one end where the catheters are attached. The other two ends of the diverting valves both connect to two separate manifolds, each with the same number of ports as diverting valves.

One manifold is assigned to the pressure source to inflate the inflatable components from a sole pressure source. A pressure gage and an open/close valve sit just behind this manifold. The pressure gauge allows the monitoring of the pressure being supplied to the inflatable components, and to shut off the pressure source when any desired pressure is reached. The second manifold is assigned to the vacuum sink where negative pressure is created to pull cleaning solution from the inflatable components to a disposal sink.

The open/close valve located behind the pressure manifold can also be closed to retain pressure inside the inflatable components. This allows any cleaning age supplied to dwell inside the inflatable component and lumen. Closing the system with this valve also allows the pressure gauge to be able to detect and measure various attributes about the inflatable components. Leaks/holes in the catheters can be detected by measuring drops in pressure, as well as the rate at which the inflatable components fill. The diverting valves also allow any combination of inflatable components to be opened to the pressure source or vacuum sink at the one time. For example, all inflatable components can be pressurized simultaneously during a cleaning cycle, one inflatable component can be pressurized and checked for leaks, a combination of inflating inflatable components and deflating can be performed simultaneously, etc.

The pressure source can be powered by various types of pumps, including centrifugal pumps, gear pumps, diaphragm pumps, peristaltic pumps, mechanical pumps, piston pumps, rotary vane pumps, and lobe pumps. These can be electrically, pneumatically, hydraulic, or manually powered.

The purpose of the pressure source for the device is to supply just enough pressure to allow the inflatable component to fully expand and its surface area to allow for optimal cleaning. High pressures are not necessary for cleaning purposes because they only add stress to the material of the inflatable component. However, high pressure pumps that allow an inflatable component catheter to reach its rated burst pressure or nominal pressure increase the ability of the system. After an inflatable component catheter is cleaned, inflating the inflatable component to its rated burst pressure in conjunction with a pressure gauge can ensure the inflatable component is free from leaks and is operating as intended. Analog and digital pressure gauges can be used to achieve the necessary pressure level, as well as monitor drops in pressure due to leaks, however computer-controlled pressure transducers allow for more pin-point accuracy as well as more advanced data collection such as the rates of pressure increase/decrease which can be characterized for more advanced leak recognition.

The vacuum sink can be driven by several types of devices including vacuum pumps, venturi pumps, liquid or steam powered jet pumps, drum pumps, diaphragm vacuum pumps, mechanical pumps, rotary vane vacuum pumps, liquid ring, vacuum pumps, and hydraulic cylinders. These pumps can be powered electrically, pneumatically, hydraulically, or steam powered. The vacuum sink does not need to achieve high negative pressures within the inflatable components. Its purpose is to remove the “contaminated” cleaning agent from the inflatable components for fresh cleaning agent to be cycled into the balloons.

When controlled by a computer, the system can also be connected to any ERP system. Bar code scanning can streamline recipe selection and pressure parameters used within the system based on the inflatable component scanned. Also detailed reports could be generated for lots and tracking purposes.

Detection Methods of full Surface Area Contact from Cleaning Solution

### Pressure Based System Logic

A version of the pressure-based system described above was assembled using electrically controlled pumps and valves controlled by a computer. The logic for this system can be seen in the flowchart in FIGS. 11A-C, where FIG. 11A is the first set of logic, followed by FIG. 11B, followed by FIG. 11C.

### Flow Rate Based System

The system described herein focuses on using pressure to determine that catheters have been inflated enough to ensure cleaning agent is contacting the surface area within the lumen and interior of the inflatable component. However, the same process can be performed by measuring flow rates instead of pressure. By characterizing the volume of the inflatable components when they are at the desired pressure to be fully inflated, a computer-controlled flow meter can accurately measure the amount of volume pumped into the inflatable components by measuring flow rate and time.

### Force Gauge System

A force gauge setup can also be used to determine that all inflatable components are fully expanded by the cleaning agent, hence, meaning that the internal surface area is coming into contact with the cleaning solution. Using a force gauge system to measure force of expansion at multiple points along the inflatable component, indicates full expansion of the inflatable component from the proximal end to distal end of the inflatable component. By characterizing the amount of force exerted by the inflatable components when fully inflated at a certain height (can be determined by dimensions of the inflatable component diameter and type of balloon material-compliant, semi-compliant, non-compliant), a force gauge positioned above the inflatable components would make a sufficient alternative to a pressure transducer or flow meter.

The height and contact points would be determined by the dimensions of the inflatable component configuration. The force readings at each contact point can be characterized by pressurizing the inflatable component to different specified pressures dictated by the cleaning process. The correlation between the forces at each contact point would indicate that the entire inflatable component is expanded via the cleaning solution, as illustrated in FIG. 4.

### Sensor Based System

This device could also be based on using sensors, for example proximity sensors. By characterizing the distance between a proximity sensor and the wall of an inflated and deflated inflatable component, a proximity sensor would provide sufficient measurement to determine if an inflatable component has inflated during the cleaning process or has completely deflated when vacuum pressure is applied, as illustrated in FIG. 5.

The inflatable components shaft would sit at a constant distance from the proximity sensors, and measurement of the distance of the walls to the sensor for each inflatable component configuration would be recorded for both an inflated inflatable component and deflated inflatable component. When these values are met, a computer would be able to determine that complete inflation/deflation has been achieved.

A very precise proximity sensor feeding information to a computer could also determine if there are leaks or pinholes present in the inflatable components. If a inflatable component is being filled with cleaning agent and never fully expands over a large amount of time, the proximity sensor could determine that a leak is present. If pressure has been applied to the inflatable component, and all valves are closed to lock pressure into the system, the proximity sensors would be able to determine if the inflatable component is deflating over time by the increasing distance of the inflatable component walls from the sensors indicating a small leak within the inflatable component.

### Machine Vision System

A machine vision system could also be used to determine that inflatable components are inflating and deflating to the proper size while being cleaned. Positioning the cameras to observe the inflatable components, and characterizing each catheter configuration by the size of the inflatable components when fully inflated and fully deflated would provide an efficient system.

The machine vision system would observe inflatable components as they are being filled and ensure that full expansion is being achieved, as well as full deflation when vacuum pressure is applied to remove cleaning agent.

A machine vision system could determine that leaks are present when inflatable components are being filled if complete expansion is not achieved over a large amount of time. Pin holes could also be detected in pressurized inflatable components by closing all valves to lock pressure within the inflatable components and measuring the inflatable component walls for a decrease in diameter over time.

### Laser Micrometer System

Detecting a full expansion of the inflatable component catheter can be achieved using a Laser Micrometer system in place. The laser micrometer uses light to accurately measure the diameters of inflatable component catheters when fully expanded and deflated. Taking multiple diameter readings of the inflatable component catheter (proximal, distal, and middle) would yield the inflatable component profile when pressurized with the cleaning solution. The appropriate measurements would be dictated by the dimensions of the inflatable component. These dimensions would be characterized by the type of inflatable component material (compliant, non-compliant, semi-compliant). Full expansion to labeled diameters would indicate that the internal surface area is being expanded by the cleaning solution. Characterization of this would be conducted using a laser micrometer and pressurizing at different values. Pressure only accumulates when there is backpressure from an enclosed system. Pressurizing different inflatable component catheters (compliant, non-compliant, semi-compliant) can be directly correlated to the diameter growth of the inflatable component to determine the proper range for contacting all the internal surface area of the catheter.

### Weight Based System

A scale could also be used to determine that inflation and deflation are occurring by characterizing the weight of the catheters when fully inflated and deflated. The appropriate weight can depend on the density of the cleaning solution and volume needed to fully inflate an inflatable component catheter. Calculations for this process are shown in Eq. 8.

$$Density of cleaning solution = m a s s / volume \qquad ( Eq . 8 )$$

First, the volume needed to fully expand the inflatable component to a desired diameter is determined. Then the equation is solved for mass as shown in Eq. 9.

$$Mass = Density * Volume \qquad ( Eq . 9 )$$

Once the mass is determined, the system can be configured to measure for that added weight, indicating that the inflatable component catheter is fully expanded and the cleaning solution is able to make contact with the full amount of surface area.

The scale would feed information to the computer when catheters are being filled to determine that enough cleaning agent has been added to the inflatable component based on the added weight. The same logic would be applied when applying vacuum pressure to remove cleaning agent. When the weight of the catheter without any cleaning agent is achieved with some tolerance, the system can determine that the inflatable component catheter is completely free from cleaning solution.

Full inflation rate is calculated using Eq. 10.

$$Full Inflation Weight = Weight of empty catheter + weight determined above ( based on how much volume is needed ) \qquad ( Eq . 10 )$$

Full deflation weight is calculated using Eq. 11.

$$Full Deflation Weight = Weight of empty catheter \qquad ( Eq . 11 )$$

Leaks in the inflatable components would be detected by measuring the weight of the catheters as they are being filled, and if the proper “filled weight” is not achieved over a long period of time a leak would be determined. The system would also detect pin holes in pressurized inflatable components by closing all valves to lock pressure within the inflatable components and detecting reductions in weight over time.

FIGS. 6A-B show catheters vertically hung from a scale. FIG. 7 shows catheters on a horizontal scale. Multiple orientations can be used to measure the weight of fully inflated devices. Within these configurations the measurements can be done in different ways.

### Hanging Method

Assuming that all inflatable component catheters are the same configuration, calculating the weight needed from the equations above can be multiplied by the number of catheters being reprocessed to measure the total added weight of the system. If inflatable component catheters are different configurations, then calculating the appropriate added weight of each configuration can be added together to calculate the Total Added Weight of the system. Lastly, this hanging method can be set up to have the capability of weighing each inflatable component catheter individually to check for leaks and ensure full expansion by cleaning solution.

### Horizontal Method

This method has the inflatable components lying flat on the surface of the scale. The total added weight is calculated in the same manner as above.

### System Configurations

One Pump Configuration with Pressure Transducer

Using the proper pump, a single pressure source pump in conjunction with additional diverting valves can be used to provide both pressure for inflating the inflatable components as well as providing vacuum pressure to pull the cleaning agent from the balloons, as illustrated in FIG. 1.

Two Pump Configuration with Pressure Transducer

As illustrated in FIG. 2, a configuration of two separate pumps as a pressure source and a vacuum sink can be used to achieve higher vacuum pulls to drain the inflatable components more quickly and efficiently, especially for high batch sizes. This would also allow simultaneous inflation and deflation of different inflatable components or manifolds with larger batch sizes.

Pump and Jet Pump Configuration with Pressure Transducer

As illustrated in FIG. 3, using an electrically or pneumatically powered pump as the pressure source to provide pressure for inflatable component inflation can also provide the pressure to drive a liquid powered venturi or jet pump. Pressurizing a venturi or jet pump provides the vacuum sink to pull contaminated cleaning agent from the inflatable component.

### Enclosable Pressurized System

For the reprocessing process the exterior of the catheters also needs to be cleaned. This device can be configured so that the catheters lay horizontally while being inflated. This would allow them to be placed in a sink so that they can be soaked or sprayed simultaneously on the exterior, while this device cleans the inner lumen and inflatable component. With the proper pump and plumbing configuration, the exterior cleaning and interior cleaning could be performed using the same pump source that is used to pressurize and inflate the inflatable components.

### Vertical Configuration for Exterior Cleaning

Configuring this device to allow the catheters to be hung vertically in a container would allow more catheters to be cleaned at once while occupying less space. Hanging them vertically also puts less stress on the shaft and connectors/hubs of the catheters as they are inflated and deflated. The chamber would allow the exteriors to be sprayed using a spray manifold while the device simultaneously cleans the inner lumens and inflatable components of the catheters. With the proper pump and plumbing configuration, the exterior cleaning and interior cleaning could be performed using the same pump source, or two separate pumps.

What is claimed is:

1. A system for cleaning a medical device that includes an inflatable component, the system comprising:
a cleaning solution source operable to contain a fluid;
at least one medical device attachment port in fluid communication with the inflatable component; and
at least one pressure source operable to move the fluid from the cleaning solution source to the at least one medical device attachment port.

2. The system of claim 1, wherein the at least one medical device attachment port comprises a cleaning valve.

3. The system of claim 2, wherein the cleaning valve is operable to direct the fluid into the inflatable component and direct the fluid out of the inflatable component.

4. The system of claim 3, the system further comprising at least one pressure transducer and/or pressure sensor operable to measure a pressure inside the inflatable component.

5. The system of claim 1, further comprising one or more fluidic paths that include an inflatable component fluidic path, a lumen fluidic path, and an exterior fluidic path.

6. The system of claim 5, wherein the inflatable component fluidic path is operable to provide the fluid to the inflatable component, the lumen fluidic path is operable to provide the fluid to one or more lumens of the medical device, and the exterior fluidic path is operable to provide the fluid to an exterior surface of the medical device.

7. The system of claim 1, wherein the at least one medical device attachment port comprises a plurality of medical device attachment ports.

8. The system of claim 7, further comprising one or more manifolds in fluid communication with the at least one pressure source, wherein the one or more manifolds are operable to direct the fluid to the plurality of medical device attachment ports.

9. The system of claim 1, the system further comprising at least one fill sensor operable to determine than an entirety of an interior surface of the inflatable component has been contacted with the fluid.

10. The system of claim 9, wherein the at least one fill sensor includes a pressure sensor, a pressure transducer, a force gauge system, a proximity sensor system, a scale, a flow rate sensor, a machine vision system, and/or a laser micrometer system.

11. A system for cleaning a medical device with an inflatable component, the system comprising:
one or more fluidic paths including an inflatable component fluidic path, a lumen fluidic path, and an exterior fluidic path;
at least one cleaning solution source in fluid communication with the one or more fluidic paths, the at least one cleaning solution source operable to contain a fluid;
at least one pressure source operable to direct the fluid to the one or more fluidic paths;
at least one medical device attachment port in fluid communication with the inflatable component fluidic path; and
one or more valves in fluid communication with the one or more fluidic paths, the one or more valves operable to direct fluid to the inflatable component fluidic path, the lumen fluidic path, and the exterior fluidic path.

12. The system of claim 11, the system further comprising at least one inflatable component fill sensor, the at least one inflatable component fill sensor operable to determine an interior surface of the inflatable component is fully contacted with the fluid.

13. The system of claim 11, the system further comprising at least one lumen pressure sensor and/or pressure transducer, the at least one lumen pressure sensor and/or pressure transducer operable to measure a pressure within a lumen of the medical device.

14. The system of claim 11, wherein the at least one medical device attachment port comprises a plurality of medical device attachment ports.

15. The system of claim 14, the system further comprising one or more manifolds in fluid communication with the one or more fluidic paths, wherein the one or more manifolds are operable to direct the fluid to the plurality of medical device attachment ports.

16. A method for cleaning a medical device with an inflatable component, the method comprising:
coupling the medical device with the inflatable component to at least one medical device attachment port;
providing, via at least one pressure source, a fluid to the inflatable component through the at least one medical device attachment port; and
monitoring, via at least one fill sensor, a level of contact of the fluid with an interior surface of the inflatable component.

17. The method of claim 16, further comprising turning off the at least one pressure source when the level of contact indicates an entirety of the interior surface of the inflatable component is contacted with the fluid.

18. The method of claim 16, further comprising determining whether the inflatable component has a leak based on a rate of change in the level of contact.

19. The method of claim 16, further comprising:
removing, via the at least one pressure source, the fluid from the inflatable component; and
monitoring, via a chemical sensor, an iodine concentration in the fluid removed from the inflatable component.

20. The method of claim 19, further comprising repeating the method when the iodine concentration indicates the inflatable component has not been sufficiently cleaned of contrast.

21. A method for cleaning one or more medical devices that include an inflatable component, the method comprising:
actuating one or more valves in communication with at least one pressure source such that an inflatable component of a medical device that is coupled with at least one medical device attachment port is in communication with the at least one pressure source and a cleaning solution source;
providing a fluid from the cleaning solution source to the inflatable component;
actuating the one or more valves such that the fluid is retained in the inflatable component for a dwell period;
actuating the one or more valves such the inflatable component is in communication with a contamination sink; and
removing the fluid from the inflatable component to the contamination sink.

22. The method of claim 21, further comprising monitoring a pressure inside the inflatable component when the fluid is retained in the inflatable component.

23. The method of claim 22, wherein when the pressure decreases by a threshold amount during the dwell period, the inflatable component is determined to have a leak.

24. The method of claim 21, wherein the one or more medical devices include a plurality of medical devices.

25. The method of claim 24, wherein the one or more valves include a plurality of valves operable to selectively supply the fluid to each of the plurality of medical devices.

26. The method of claim 22, wherein the fluid includes one or more of micro-pellets, microbeads, microparticles, abrasive particles, and/or microbubbles operable to provide abrasive cleaning to the inflatable component and/or a lumen of the one or more medical devices.
